(19) United States
(12) Patent Application Publication
(10) Pub. No.: US 2025/0313792 A1
(43) Pub. Date: Oct. 9, 2025

(54) Devices, systems, and methods for culturing cells in a 3-dimensional (3-D) arrangement

(71) Applicant: Northeastern University, Boston, MA (US)

(72) Inventors: Kyla Kaiser, Wakefield, MA (US); Abigail N. Koppes, Charlestown, MA (US); Ryan A. Koppes, Charlestown, MA (US); Jessica Snyder, Malden, MA (US); Bryan Schellberg, Pawtucket, RI (US); Adam James Bindas, Utrecht (NL)

(21) Appl. No.: 19/170,508

(22) Filed: Apr. 4, 2025

Related U.S. Application Data
(60) Provisional application No. 63/575,401, filed on Apr. 5, 2024.

(51) Int. Cl. C12M 1/12; C12M 1/00; C12N 5/00

(52) U.S. Cl. CPC C12M 25/02; C12M 29/00; C12N 5/0062

(57) ABSTRACT

Devices, systems, and methods for culturing cells in a 3-dimensional (3-D) arrangement are disclosed. A microfluidic device includes a plurality of layers including a first layer defining a first chamber for holding a first cell culture, a second layer defining a second chamber for holding a second cell culture, and channel layers defining channels. The first and second chambers are fluidically coupled and enable the first cell culture and the second cell culture to grow in the 3-D environment. A porous membrane is positioned between the first and second layers to enable interfacing between the first cell culture and the second cell culture. The channels are fluidically coupled with the first chamber or the second chamber to enable passage of fluids with respect to the cells cultured. The microfluidic device enables development of more physiologically accurate models of complex tissues or organs for basic science research or drug development.

### RELATED APPLICATION

This application claims the benefit of U.S. Provisional Application No. 63/575,401, filed on Apr. 5, 2024. The entire teachings of the above application are incorporated herein by reference.

### GOVERNMENT SUPPORT

This invention was made with government support under Grant Number CBFT-2045906 awarded by the National Science Foundation, and Grant Number 1R35GM142741-01 awarded by the National Institutes of Health. The Government has certain rights in the invention.

### BACKGROUND

Organ on a chip (OOC) technology may enable the development of microfluidic systems that mimic the physiological and mechanical environments of tissues or organs of living organisms. Such devices may be helpful for, as non-limiting examples, benchtop assays, drug development, or basic discovery of human and animal physiology. However, widespread adoption of OOC technology, including recent advancements thereof, may be limited due to challenges regarding robustness or reliability of a given OOC device and repeatability of experiments or assays performed thereon. Furthermore, recapitulating complex, 3-D cellular architectures found in the tissues of humans and other living organisms, which may be important for modeling and understanding tissue function, may be difficult.

### SUMMARY

The embodiments described herein may be helpful for more accurately modeling complex tissue structures in a microfluidic device and for enabling more efficient, high-throughput evaluation of a microphysiological system including the complex tissue structures.

In an example embodiment, a fluidic device for culturing cells in a three-dimensional (3-D) arrangement includes a plurality of layers. The plurality of layers includes a first layer configured to define a first chamber for holding a first cell culture and a second layer configured to define a second chamber for holding a second cell culture. The first layer and the second layer are in coupled arrangement to fluidically couple the first chamber and the second chamber and to enable the first cell culture and the second cell culture to grow in the 3-D arrangement. A porous membrane is positioned between the first and the second layer and is configured to enable interfacing between at least a portion of the first cell culture and at least a portion of the second cell culture through the porous membrane. The plurality of layers further includes one or more channel layers configured to define one or more channels. Each channel layer is in coupled arrangement with the first layer or the second layer to fluidically couple each channel with at least one of the first chamber or the second chamber, wherein the one or more channels are configured to enable passage of one or more fluids with respect to the cells cultured in the 3-D arrangement.

The plurality of layers can be configured to define one or more ports fluidically coupled to the one or more channels, the first chamber, or the second chamber. The plurality of layers can further include a reservoir layer configured to define at least one media reservoir. A given media reservoir can be fluidically coupled with a channel of the one or more channels through a port of the one or more ports. The given media reservoir can be configured to dispense the one or more fluids into the channel based on an orientation of the fluidic device. Dispensing of the one or more fluids can impart shear stress on the first cell culture or the second cell culture. For example, the fluidic device may be positioned on a platform positioned at an angle and gravity may drive passage of the one or more fluids through the channel. In some embodiments, the first cell culture or the second cell culture may be a monolayer of cells and the shear stress may enable maintaining of cell phenotypes of the monolayer of cells.

A port of the one or more ports can be fluidically coupled with a pump. The pump may be configured to supplement media continuously or to cause the passage of the one or more fluids through a channel of the one or more channels. The media may be, for example, cell culture media.

The one or more channel layers can include a first channel layer configured to define a first channel fluidically coupled to the first chamber and a second channel layer configured to define a second channel fluidically coupled to the second chamber. The one or more ports can include at least one port fluidically coupling the first channel to a given surface of the fluidic device and at least a second port fluidically coupling the second channel to the given surface of the fluidic device. Such a configuration may enable ease of access to channels from a single given surface and may facilitate performing of assays within the fluidic device, for example, transepithelial electrical resistance assays.

The plurality of layers of the fluidic device can include an additional porous membrane configured to be selectively permeable. The additional membrane can be positioned between a channel of the one or more channels and the first layer or the second layer. The permeability of the additional porous membrane may be determined by a size of pores in the porous membrane.

The fluidic device can include the first cell culture and the second cell culture. The first cell culture and the second cell culture can be held in the first chamber and the second chamber, respectively.

The plurality of layers of the fluidic device can be configured to enable imaging of cells in situ. For example, a given layer of the plurality of layers may be composed of a transparent material, e.g., poly (methyl 2-methylpropenoate. The plurality of layers may also include a glass layer. The plurality of layers may enable imaging of a given layer of cells, for example, the first cell culture, the second cell culture, a depth-resolved layer of the first cell culture, or a depth-resolved layer of the second cell culture.

A given layer of the plurality of layers can be bonded to another layer of the plurality of layers. For example, the given layer can be adhesively bonded to the another layer using adhesive tape. Other examples of bonding methods may include chemical bonding or thermal bonding. The plurality of layers may be bonded together and incorporated into a single plate, apparatus, or device.

A high throughput fluidic system for culturing cells in a 3-D arrangement can include the plurality of layers described herein. The first chamber defined by the first layer, the second chamber defined by the second layer, and the one or more channels defined by the one or more channel layers can be a first fluidic unit. The plurality of layers can be further configured to define at least one additional fluidic unit.

The plurality of layers of the fluidic system can be configured to define one or more ports fluidically coupled to the one or more channels, the first chamber, or the second chamber of a given fluidic unit of the first fluidic unit or the at least one additional fluidic unit. The plurality of layers can further include a reservoir layer configured to define a plurality of media reservoirs. A given media reservoir can be fluidically coupled to a given port of the one or more ports and configured to dispense a fluid into a given channel of the one or more channels based on an orientation of the high throughput fluidic system.

A given fluidic unit of the first fluidic unit or the at least one additional fluidic unit can be fluidically independent of another fluidic unit of the first fluidic unit or the at least one additional fluidic unit.

In another example embodiment, a physiological fluidic system with cells cultured in a three-dimensional (3-D) arrangement includes a first layer configured to define a first chamber with a first cell culture, and a second layer configured to define a second chamber with a second cell culture. The first layer and the second layer are in coupled arrangement to fluidically couple the first chamber and the second chamber and to enable the first cell culture and the second cell culture to grow in the 3-D arrangement. A porous membrane is positioned between the first layer and the second layer and configured to enable interfacing between at least a portion of the first cell culture and at least a portion of the second cell culture through the porous membrane. The physiological fluidic system further includes one or more channel layers configured to define one or more channels. The one or more channel layers are in coupled arrangement with the first layer or the second layer to fluidically couple the one or more channels with the first chamber or the second chamber and the one or more channels are configured to enable passage of one or more fluids with respect to the cells cultured in the 3-D arrangement. The first layer, the second layer, the one or more channel layers, and the porous membrane may be bonded together or otherwise incorporated into a single plate, device, or apparatus.

The first cell culture or the second cell culture can include different populations of cells. The cells may typically grow in 3-D arrangements under physiological conditions. For example, one of the first cell culture or the second cell culture can include epithelial cells or endothelial cells and the other can include neuronal cells.

The physiological fluidic system can further include a structural hydrogel for the first cell culture or for the second cell culture in the second chamber.

In another example embodiment, a method for culturing cells in a 3-D arrangement in a fluidic system includes seeding a first cell culture in a first cell chamber and seeding a second cell culture in a second cell chamber. The first cell chamber and the second cell chamber are fluidically coupled and separated by a porous membrane, and the first cell chamber and the second cell chamber are further positioned to enable the first cell culture and the second cell culture to grow in the 3-D arrangement. The method further includes culturing the first cell culture and the second cell culture to enable interfacing of at least a portion of the first cell culture and at least a portion of the second cell culture through the porous membrane. The method still further includes causing passage of the fluid with respect to the cells cultured in the 3-D arrangement. The first cell chamber and the second cell chamber may be incorporated into a single plate, device, or apparatus.

The passage of the fluid can include causing a flow of the fluid at a surface of at least a portion of the first cell culture or at least a portion of the second cell culture. Causing the flow of the fluid can include utilizing a pump or a gravity-driven design. The flow of the fluid can cause shear stress at the surface.

The first cell culture can be seeded and cultured for a period of time prior to seeding of the second cell culture. In such a way, cell cultures that grow at different rates or that require maturation in different environments can be co-cultured.

As described herein, in an experimental embodiment, microfluidic devices and microphysiological systems may be useful for developing more realistic embodiments of physiological tissues or organ systems. For example, enteric neurons may be critical in maintaining organ homeostasis within the small intestine, and their dysregulation may contribute towards gastrointestinal disorders and neurodegenerative diseases. Current in vitro models may lack enteric innervation, which may limit basic discovery and disease modeling research. Example embodiments of microfluidic devices described herein, including high-throughput variations, may enable culturing of a primarily epithelial monolayer interfacing directly with encapsulated primary enteric neurons. Furthermore, design of the microfluidic device may mimic shear stress of physiological intestines by introducing flow, e.g., gravity-driven flow, of a fluid with respect to the monolayer.

In example embodiments of a microphysiological system with co-cultured epithelium and enteric neurons, intestinal and neural tissue exhibited expected morphologies. Neural gene upregulation in the epithelium may suggest RNA contamination from proximal enteric neurons extending neurites toward the epithelial monolayer. With an enteric nervous system (ENS), example results showed that barrier integrity significantly increased for both transepithelial electrical resistance (TEER) and permeability assays, a 1.25-fold greater resistance and 10% lower permeability as compared to epithelium cultured alone. Presence of the ENS resulted in a significant (1.4-fold) reduction in epidermal growth factor (EGF). Additionally, example embodiments enable that several key epithelial genes are compared between duodenal tissue and epithelial monolayers with and without neurons present. Results may demonstrate changes in cytokine gene expression and WNT pathways, highlighting innervation may be helpful for creating more biomimetic and physiologically relevant in vitro models.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 diagrammatically illustrates in a top-down view an example embodiment of an assembled microfluidic device for culturing cells in a 3-D arrangement.

FIG. 2 illustrates in an orthogonal view the example embodiment of the assembled microfluidic device of FIG. 1 for culturing cells in a 3-D arrangement.

FIG. 3 illustrates an exploded diagram of an example embodiment of a microfluidic device for cultures cells in a 3-D arrangement.

FIG. 4 illustrates an example embodiment of a high-throughput microfluidic system. An inset illustrates a fluidic unit of the high-throughput microfluidic system, which may be similar to the microfluidic device of FIG. 3.

FIG. 5 diagrammatically illustrates a gastrointestinal system (left), including a cross-section of a bulk small intestine (middle) and a cross-section of a microscopic small intestine (right).

FIG. 6 diagrammatically illustrates a reductionist model of an innervated small intestine, the model being used as a blueprint of a microphysiological system by an example embodiment.

FIG. 7 illustrates an example embodiment of a high-throughput microfluidic system including 12 independent culture systems, including a portion of an apical media channel and a basal media chamber.

FIGS. 8A-8C illustrate gravity-driven flow across epithelial cells according to an example embodiment.

FIG. 9 illustrates a seeding timeline for an example embodiment of a microphysiological system with primary rat cells and enteric neurons.

FIGS. 10A, 10B, 10C, and 10D illustrate brightfield images taken at days 1, 2, 4, and 6, respectively, of epithelial crypt cells isolated from a duodenum and expanded as intestinal organoids in a 3D culture environment prior to use in experiments, according to an example embodiment.

FIGS. 11A and 11B illustrate brightfield images of an epithelial monolayer and a plane of a gel containing enteric neurons, respectively, before fixation, according to an example embodiment.

FIGS. 11C and 11D illustrate immunostained layers of an epithelial monolayer and a plane of a gel containing enteric neurons, respectively in a microphysiological system, according to an example embodiment.

FIG. 12A illustrates an orthogonal maximum intensity projection showing layers of a culture, including beta III tubulin identifying neurons and ZO-1 identifying epithelial cells, according to an example embodiment.

FIG. 12B illustrates a 3-D surface plot showing proximity and depth of different cell types in an example embodiment of a culture system.

FIGS. 13A-13D illustrate immunocytochemistry images of cultures of rat neonatal enteric neurons under standard culture conditions according to an example embodiment.

FIGS. 14A and 14B illustrate plots of a percentage of neurons expressing vasoactive intestinal peptide or choline acetyltransferase in cultures similar to those of FIGS. 13A-13D.

FIG. 15 illustrates a plot of a percentage of neurons expressing vasoactive intestinal peptide or choline acetyltransferase out of a total number of cells in cultures similar to those of FIGS. 13A-13D.

FIG. 16A illustrates a schematic of a transepithelial electrical resistance (TEER) assay in an example embodiment of a microfluidic device.

FIGS. 16B and 16C illustrate results of a transepithelial electrical resistance assay similar to the TEER assay schematically illustrated in FIG. 16A, according to an example embodiment.

FIG. 17A illustrates a schematic of an apparent permeability assay in an example embodiment of a microfluidic device.

FIGS. 17B and 17C illustrate results of an apparent permeability assay similar to the apparently permeability assay illustrated in FIG. 17A, according to an example embodiment.

FIGS. 18A and 18B illustrate microscopy images of monolayers in example embodiments of a microphysiological device.

FIG. 19 illustrates a plot of normalized area covered for microscopy images similar to the microscopy images of FIGS. 18A and 18B according to an example embodiment.

FIG. 20 illustrates a plot of concentrations of mucin 2 in cell cultures according to an example embodiment.

FIG. 21 illustrates a plot of concentrations of epidermal growth factor in cell cultures according to an example embodiment.

FIG. 22 illustrates a heat map showing differential gene expression in log 2-fold change of experimental groups including freshly isolated duodenal crypts, MPS epithelium monoculture, or MPS epithelium in co-culture with enteric neurons, according to an example embodiment.

FIGS. 23A-23F illustrate plots of relative gene expression of different categories of genes for the experimental groups of FIG. 22.

FIG. 24 illustrates a plot of top 20 pathways enriched by presence of neurons in in vitro co-cultures, according to an example embodiment.

### DETAILED DESCRIPTION

A description of example embodiments follows.

The behavior and physiology of cells or groups of cells may be strongly influenced by their microenvironment, organization, and 3-dimensional (3-D) architecture, for example, in tissues and organs of multicellular organisms.

An example organ with complex 3-D cellular architecture is the human or animal gastrointestinal (GI) tract, which forms a selectively permeable barrier, allowing nutrient transport into the host while keeping pathogens out. The gut is host to millions of harmful and symbiotic microorganisms that the intestinal epithelium must keep separate from the inner circulatory system. The integrity of this epithelial barrier is essential for systemic health. A compromised intestinal barrier is implicated in several GI disorders due to bacteria and toxins infiltrating the tissue and causing inflammation. These disorders may include the discernible irritable bowel syndrome (IBS) and Inflammatory Bowel Disease (IBD) and are also comorbid with several diseases including depression and hypertension, suggesting that gut health may impact the entire body. In addition to the epithelial barrier, enteric neurons of the enteric nervous system (ENS) may play a critical role in maintaining organ homeostasis within the small intestine, and their dysregulation may be implicated in gastrointestinal disorders and neurodegenerative diseases.

However, underlying mechanisms of GI disorders may remain elusive due to the complexity of entire organisms, especially considering the interplay between cognitive and gut health during in vivo investigations. Therefore, a reductionist approach utilizing in vitro models may be helpful for understanding the regulation of barrier function and the impact the gut may have on human systemic health. Furthermore, the reductionist approach may enable at least partial recapitulation of the structure and function of a GI system in a microphysiological system (MPS).

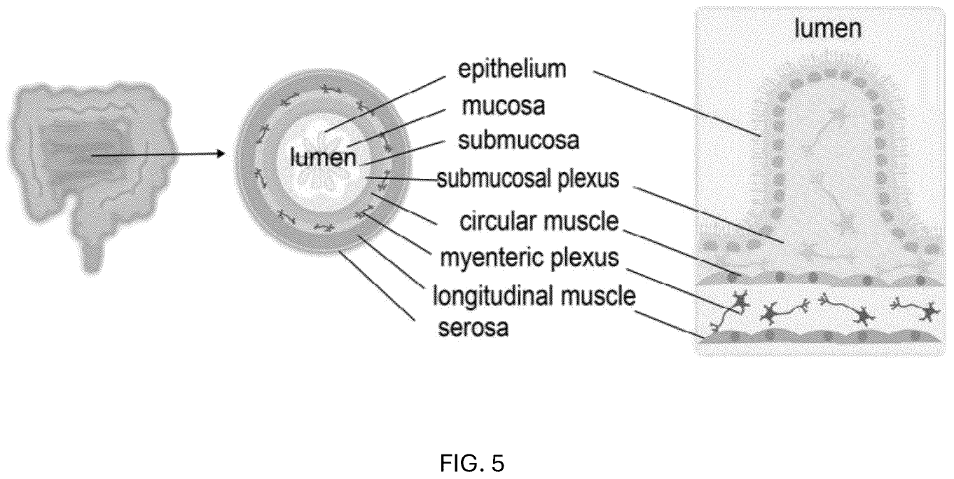

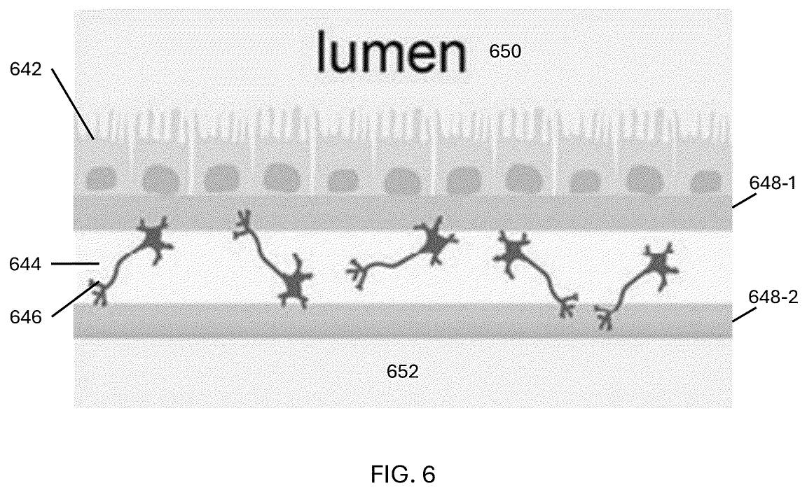

FIGS. 5 and 6, described below, provide illustrations of the GI system and of an example reductionist model thereof.

Microphysiological systems, or organ chips, have been developed for the gut using various materials and architectures, such as human tissue, inflammatory compounds, shear stress to model peristalsis, compositions of the microbiome, and villi-like structures, that may be helpful for providing physiological relevance. Due to advantages of MPSs over in vivo models, MPSs may be used in a broad range of research applications from drug delivery to disease progression. Increasingly expanded complexity of gut-on-a-chip systems, including physiological components such as shear stresses via medium perfusion, have shown faster differentiation and increased mucus production compared to traditional 2D culture methods. Some MPSs may also support analysis in situ and in real-time, for example, using standard light microscopy and experiments evaluating barrier function and permeability, which are especially relevant in modeling epithelial dysfunction such as in IBD. Transepithelial electrical resistance (TEER) and apparent permeability measurements of fluorescent molecule diffusion are example non-destructive methods that may be used to assess barrier integrity of live epithelial monolayers on chip. The health of these epithelial barrier properties may be pivotal for studying microbiome interactions and drug absorption. While such techniques may be readily implemented in Transwell cultures, the difficulty and cost of integrating such sensing techniques for barrier function in MPSs may limit broader adoption of the MPSs.

Current gut epithelium MPSs may require cell lines, isolated primary cells, or induced stem cells. The human colorectal adenocarcinoma cancer cell line, Caco-2, is frequently used as an epithelium model, with maturation of the cancer cell line leading to tight junction formation. However, using Caco-2 cells in MPSs to model the gut may have limitations as they only comprise the absorptive enterocyte phenotype, lacking the heterogeneous population found in vivo. The use of primary epithelium cell models, which may include seeding monolayers of epithelial cells isolated from expandable intestinal epithelial organoids which contain intestinal stem cells and epithelial cell subtypes responsible for neural, immune, and microbial cell interactions, may provide a more physiologically relevant model of the gut. Specifically, these diverse populations include absorptive enterocytes, mucus-producing goblet cells, and secretory enteroendocrine and Paneth cells. Primary epithelium models are only obtainable through rodent tissue or human biopsy samples. However, in vitro, these populations proliferate easily, which may allow sizable stocks of the cells to be cultured or cryopreserved for up to several years. These heterogeneous epithelium populations may more closely resemble the human gut than traditional immortalized cancer cell lines and may be needed to explore the role of the autonomic nervous system (ANS) in barrier function.

The degree and synaptic organization of the enteric nervous system on the function of each of these epithelial populations is not fully understood. Sensations and coordination of gastrointestinal activities may be facilitated by the underlying neurons of the gut, the ENS, as well as branches of the autonomic nervous system composed of plexi, ganglia, spinal cord, and cranial nerves. Greater in total neuron numbers than the spinal cord, enteric neurons may inform the epithelium's proliferation through epidermal growth factor (EGF) signaling in enterocytes, the most abundant epithelial subtype. The enteric nervous system may further aid in preventing microbial infection by promoting goblet cell mucus production, resulting in a thicker, stronger barrier. Enteric neurons may also play a role in immunity, producing cytokines (IL-6, IL-18) as well as sensing inflammatory cytokines (TNF-α, TGF-β, IL-4). Epithelial-enteric neuron communication has been found to occur through soluble neuronal mediators in addition to direct synapsing onto enteroendocrine cells. One example of paracrine signaling in the gut occurs through Vasoactive Intestinal Peptide (VIP) released by VIPergic enteric neurons. VIP may promote proliferation of the epithelial cells, improves the barrier, and increases secretion. Conversely, acetylcholine (Ach), released by cholinergic enteric neurons, may increase permeability and decrease proliferation of the epithelium, which may suggest that an imbalance between these two neurotransmitters can lead to barrier dysfunction.

In some embodiments of the present invention, microfluidic devices may be configured to create an MPS including epithelial cell lines and enteric neurons. In such a system, specific 3-dimensional (3-D) architectures of different cell types or cultures may enable more accurate modeling of physiological behavior and function of gut cell types.

In addition to incorporating enteric neurons within an MPS, some embodiments of the present invention may be engineered to address many of the current limitations of gut-chip designs, for example, low sample throughput, high media consumption, and reliance on pumps along with their required fluidic connectors to induce shear. A specific embodiment of an MPS design described herein may support primary epithelial monolayers and 3D enteric neuron encapsulation in up to twelve independent samples, all within a standard tissue culture plate footprint (≈85×127 mm). Uniform pulsatile flow/shear across each sample may be achieved using media reservoirs that support pumpless, gravity-driven flow when placed on a laboratory rocker to induce physiologically relevant shear stresses. The device may be assembled with a glass base to enable high resolution microscopy through the entire height of the MPS so that both the epithelial and neuronal populations can be monitored during the experimental time course and analyzed for endpoint immunohistochemical characterization.

A presence of enteric neurons in co-cultures with epithelial cells may alter epithelial differentiation and maturation timeframe compared to epithelial-only cultures. Using example embodiments of the present invention, an impact of enteric neurons on the epithelial barrier stability may be studied with permeability assays, e.g., TEER and lucifer yellow diffusion. Enzyme-linked immunosorbent assay (ELISA) assays and epithelial RNA sequencing may be carried out as steps toward understanding underlying biological mechanisms of ENS regulated barrier function in vitro and comparing culture gene expression to duodenal tissue. Results of example embodiments of microphysiological systems may indicate that enteric neurons positively influence epithelial barrier strength, alter growth factor signaling, and change gene expression.

### Co-Cultures of Epithelial Cells and Enteric Neurons are Supported by a Cost Effective, Pumpless, High Throughput MPS

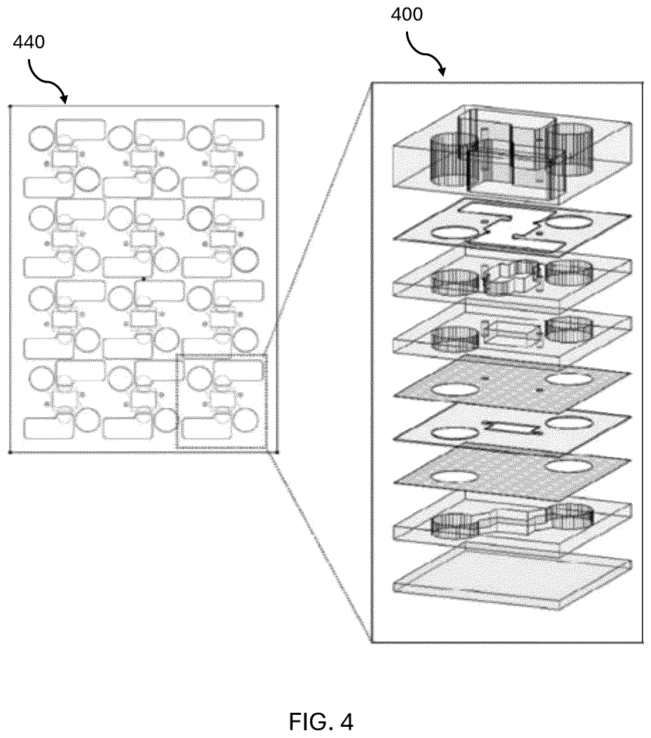

As described herein, example embodiments of the present invention may include a microfluidic device for creating an innervated microphysiological system of primary duodenal epithelium to investigate the role of enteric neurons on primary epithelial cell permeability. The microfluidic device platform may represent a departure from traditional MPSs fabricated via PDMS, which may be limited in design/redesign and may often be cost prohibitive at $150-500 per design. Adding neurons in the innervated MPS may be critical for developing more complex and biomimetic organ chip devices and may further enable MPSs to become the standard for scientific research, including applications in studying developmental biology, drug delivery, and disease progression. In another example embodiment of the present invention, a high-throughput microfluidic device may include a 12-unit MPS, as described herein with respect to FIG. 4.

Example embodiments of an MPS, which may include a single fluidic unit or multiple fluidic units in a high-throughput embodiment, may be fabricated using laser-cut thermoplastic layers with a method previously described in U.S. Pat. No. 11,351,538 B2, by Hosic et al. A method of assembly of a microfluidic device or organ on a chip system are described in published PCT Application No. WO2024/112835A2, by Koppes et al. In a specific embodiment, polymethyl methacrylate (PMMA) and polyethylene terephthalate (PET) layers may facilitate an oxygen impermeable environment.

An example 12-sample chip, as described herein with respect to FIG. 4, may be produced in under a couple of hours for $21 dollars, or less than $2 per unit or sample. The scaled-up, 12-sample chip device may support 12 co-culture chambers on a single 76 mm by 101 mm chip, which fits well within a standard well plate footprint with dimensions of approximately 127 mm by 85 mm. This example high-throughput embodiment may address several current challenges in the robustness, reproducibility, and reliability of organ-chip platforms. The example embodiment of the MPS features a top chamber on a permeable membrane, which may allow epithelial monolayer adhesion and polarization, and a lower chamber, which may support a 3D culture below the membrane where enteric neurons may be seeded, for example, within a Matrigel and collagen solution.

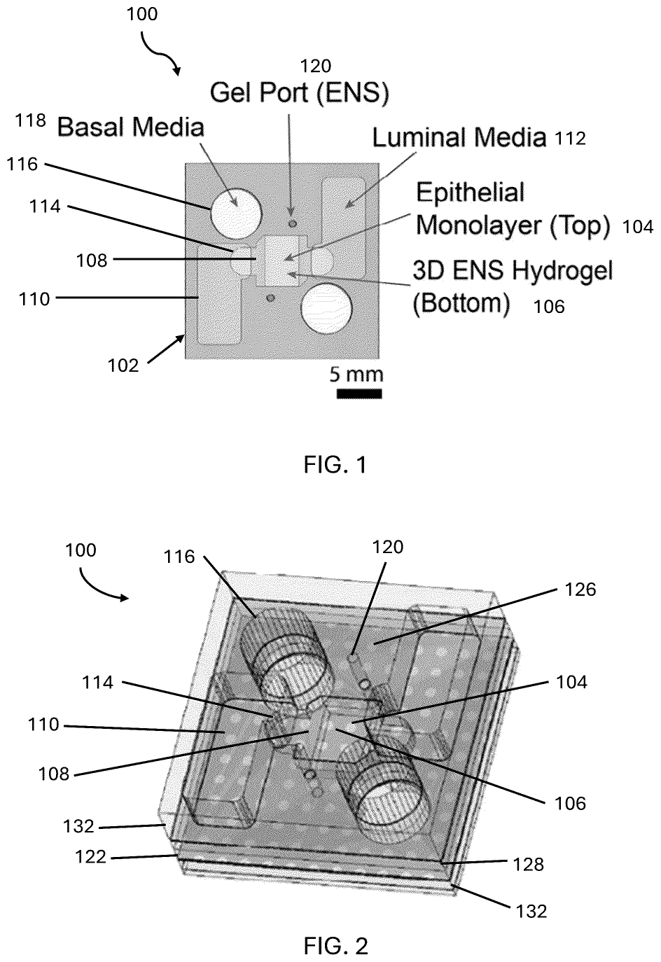

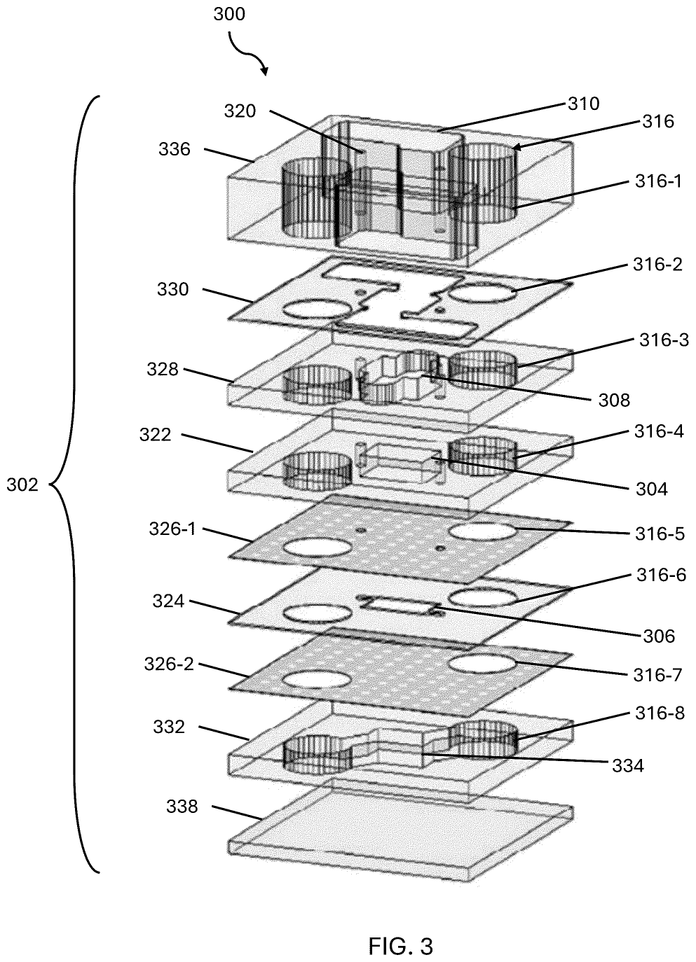

FIGS. 1 and 2 illustrate a top-down view and an orthogonal view, respectively, of an example embodiment of a microfluidic device 100 for culturing cells in a 3-D arrangement. The microfluidic device 100 includes a plurality of layers 102, which may be bonded together. The plurality of layers 102 includes a first layer 122 configured to define a first chamber 104 for holding a first culture of cells, which may include a culture comprising an epithelial monolayer. The plurality of layers 102 further includes a second layer (not visible but described herein with reference to FIG. 3) configured to define a second chamber 106 for holding a second culture of cells, which may include cells of an enteric nervous system. The first layer 122 and the second layer are in coupled arrangement to fluidically couple the first chamber 104 and the second chamber 106 this coupled arrangement enables the first cell culture and the second cell culture to grow in the 3-D arrangement. The plurality of layer 102 includes a porous membrane 126 configured to enable interfacing between at least a portion of the first cell culture and at least a portion of the second cell culture through the porous membrane. The plurality of layers 102 further includes one or more channel layers configured to define one or more channels. Each channel layer of the one or more channel layers is in coupled arrangement with the first layer or the second layer to fluidically couple the one or more channels with at least one of the first chamber 104 or the second chamber 106. The one or more channels are configured to enable passage of one or more fluids with respect to the cells cultured in the 3-D environment.

In this example embodiment, a first channel layer 128 defines a first channel 108 fluidically coupled to the first chamber 108. The first channel 108 is configured to enable passage of a medium, for example, a luminal media 112, with respect to the cells of the first chamber 104, for example, the epithelial cell culture. The plurality of layers further includes a reservoir layer 136 configured to define one or more medium reservoirs, e.g. 110, which may be configured to dispense the luminal media 112 into the first channel 108 through a first port 108, defined by the plurality of layers 102. In addition, a second channel layer 132 defines a second channel (not shown but described herein with respect to FIG. 3) fluidically coupled with the second chamber 106. The second channel is configured to enable a passage of a medium, for example, a basal media 118, with respect to the second cell culture, for example, the cells of the ENS. The basal media 118 may be dispensed into the second channel through a second port 116 defined by the plurality of layers and fluidically coupled with the second channel. The microfluidic system 100 further includes gel ports, e.g., 120, defined the plurality of layers 102. The gel ports, e.g., 120, are fluidically coupled to the second chamber 106 and enable seeding of the second chamber 106 with the cells of the ENS.

FIG. 3 illustrates an exploded diagram of an example embodiment of a microfluidic device 300 for cultures cells in a 3-D arrangement. The microfluidic device 300 may be similar to the microfluidic device 100 described herein with reference to FIGS. 1 and 2 and similar components are labeled with corresponding reference numbers incremented by 200. The microfluidic device 300 includes a plurality of layers 302. The plurality of layers includes a first layer 322 configured to define a first chamber 304 for holding a first cell culture and a second layer 324 configured to define a second chamber 306 for holding a second culture of cells. A first porous membrane 326-1 is positioned between the first layer 322 and the second layer 324 and is configured to enable interfacing between at least a portion of the first cell culture and at least a portion of the second cell culture through the first porous membrane 326-1. The plurality of layers further includes a first channel layer 328 configure to define a first channel 308 and a flow inlet spacer layer 330. A reservoir layer 326 is configured to define one or more reservoirs, e.g., 310. The flow inlet spacer layer 330 may be configured to control dispensing of a medium from the one or more reservoirs, e.g., 310, into the first channel 308. The plurality of layers further includes a second channel layer 332 in coupled arrangement with the second layer 324 and configured to define a second channel 334 fluidically coupled to the second chamber 306. The second channel is configured to enable passage of a second medium with respect to the second cell culture. A second porous membrane 326-2 is positioned between the second layer 324 and the second channel layer 332. The plurality of layers 302 further includes a glass slide 338, the glass slide 338.

The plurality of layers 302 is configured to define one or more ports. As an example, the port 316 fluidically couples to the second chamber to a given surface of the microfluidic device 300 and is configured to enable dispensing of a fluid into the second chamber 316 from the given surface of the microfluidic device. To enable fluidic couple of the given surface to the fluidic device, a subset of the plurality of layers 302 are configured to define sections of the port 316. The sections of the port are defined by the reservoir layer 336 (316-1), the spacer layer 330 (316-2), the first channel layer 328 (316-3), the first layer (316-4), the first membrane 326-1 (316-5), the second layer 324 (316-6), the second membrane 326-2 (316-7), and the second channel layer 332 (316-8).

Other ports of the one or more ports, for example, the gel port 320, may be defined by a different subset of the plurality of layers. The gel port 320 is fluidically coupled to the second chamber, which is positioned, with respect to the given surface, above the second channel layer 332 and the second membrane 326-2. As such, the second channel layer 332 and the second membrane 326-2 are not configured to define a section of the gel port 320.

A high-throughput microfluidic system may comprise a plurality of microfluidic devices, such as device 100, 300, which may be arranged in an array and may be supported on a common substrate, e.g., a glass plate or another support structure.

FIG. 4 illustrates an example embodiment of a high-throughput microfluidic system 440. The high-throughput microfluidic system 440 includes a plurality of layers defining twelve fluidic units arranged in a 3-by-4 array. A given fluidic unit 400 of the twelve fluidic units, as illustrated by an inset in FIG. 4, may be defined by the plurality of layers and may be similar to the microfluidic device 300 described herein with reference to FIG. 3.

FIG. 5 diagrammatically illustrates a gastrointestinal system (left), including a cross-section image of a bulk small intestine (middle) and a cross-section image of a microscopic small intestine (right). The cross-section images illustrate the complex 3-D structure of the small intestine, which may generally include the lumen, mucosa, submucosa, muscle layer, and serosa. Each layer serves an important purpose in the structure and function of the small intestine and may be sub-divided. The mucosa includes an epithelium that may line the mucosa and may interface with the lumen of the small intestine. The submucosa may include the submucosal plexus and may include neurons of the ENS. The muscle layer may include a circular muscle layer, a longitudinal muscle layer, and the myenteric plexus, which may include further neurons of the ENS.

FIG. 6 diagrammatically illustrates a reductionist model of an innervated small intestine, the model being used as a blueprint of a microphysiological system by an example embodiment. The reductionist model includes a co-culture of an epithelial monolayer 642 and enteric neurons 646 separated by a first membrane 648-1. The enteric neurons 646 may be cultured in an extracellular matrix 644, for example, a hydrogel-based material. The epithelial monolayer 642 also interfaces with a lumen 650 that may enable a passage of a liquid. The passage of the liquid may introduce shear stress on the epithelial monolayer 642 similar to that found in a small intestine. The enteric neurons 646 and the extracellular matrix 644 may rest between the first membrane 648-1 and a second membrane 648-2, and channel 652 may be disposed on the other side of the second membrane 648-2 from the enteric neurons 646 and the extracellular matrix 644. The channel 652 may be configured to enable a passage of a neuron medium.

FIG. 7 illustrates an example embodiment of a high-throughput microfluidic system similar to the high-throughput microfluidic system 400 described herein with respect to FIG. 4. FIG. 7 further illustrates addition of media in channels of the high-throughput microfluidic system. A dark gray color in each fluidic unit indicates a first media added into a first channel, which may be an apical media channel, of a given fluidic unit and light gray color indicates a second media added into a second channel, which may be a basal media channel, of the given fluidic unit.

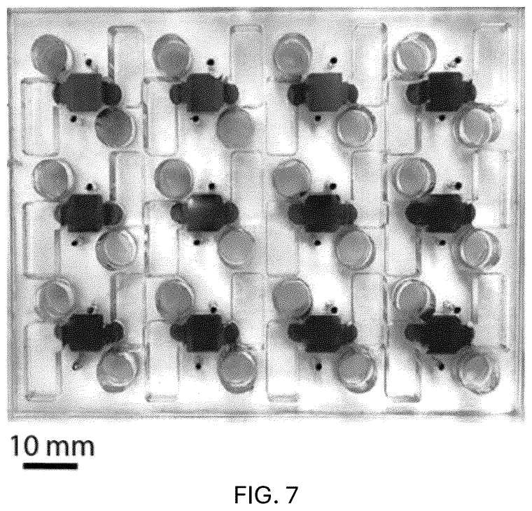

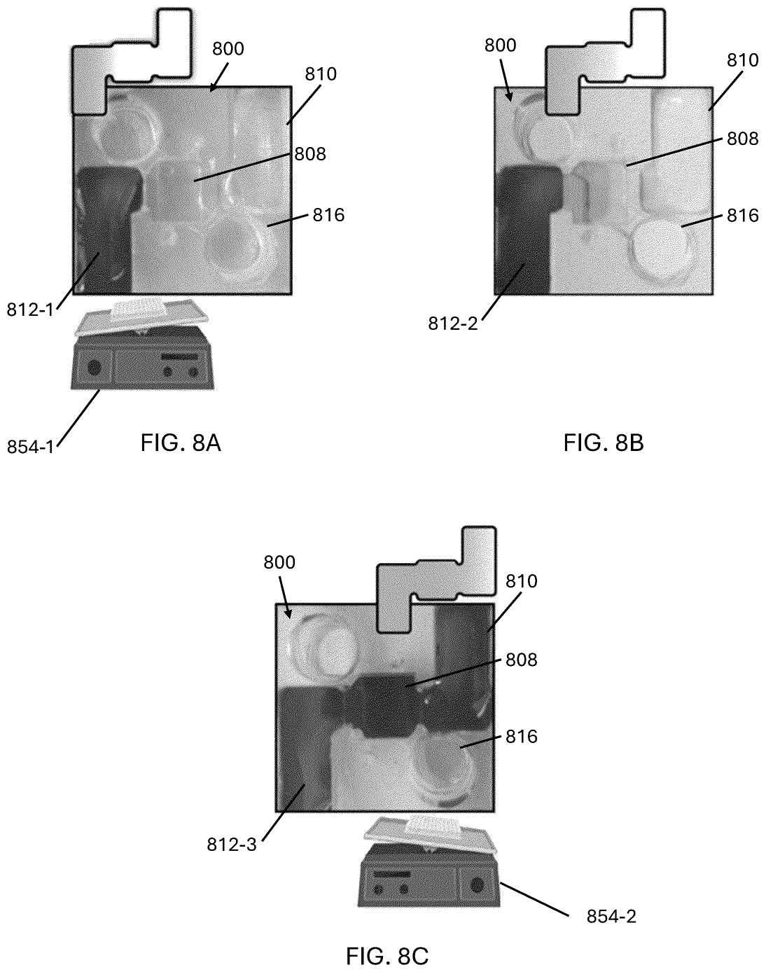

As described herein with respect to FIG. 3, the microfluidic device 300 includes the reservoir layer 336 configured to define one or more reservoirs, e.g., 310. In a specific embodiment, a microphysiological system may be designed with two large, opposing media chambers to enable application of relevant shear stress in a pump-free design. Such a design may increase robustness and ease-of-use of the MPS. Pumpless flow may be incorporated into the device via rocking to make a model more biologically relevant than static cultures as epithelial cells undergo shearing in the process of digestion and absorption. Flow of medium across the epithelial monolayer may be accomplished with a standard platform rocker, described herein with reference to FIGS. 8A-8C. In some embodiments, a large plate design may ease alignment with an axis of rotation of a rocker and ensure application of flow across all MPSs in a high-through microfluidic device. Flowing liquid induced by the rocker may create a more biomimetic environment for intestinal cells than a static culture and may avoid the use of bulky pumps and tubing that are often used in microfluidic devices to obtain shear stress.

In an example embodiment, tilt angle and speed may be adjusted to achieve pulsatile shear stress in the physiological range of 0.002-0.08 dyne cm−1. In the example embodiment, shear stress was calculated to be 0.0683 dyne cm−2 across the middle of the cell culture chamber when the rocker was at a maximum tilt angle of 2 degrees and rocking at 10 rpm.

$${θ}_{0} = 2 ( \frac{{h}_{0}}{L} ) \qquad Equation 1$$

$${τ}_{ω} = μ \frac{\partial u}{\partial x} {\vert}_{x = 0} = \{ \begin{matrix}\frac{3 {πμθ}_{ma x} x ( L - x )}{{T [ {h}_{0} cot ( θ ) + \frac{L}{2} - x ]}^{2} {sin}^{2} ( θ )} cos ( \frac{2 π t}{T} ) , θ \leq {θ}_{0} \\ \frac{3 {πμθ}_{ma x} x}{T ( \sqrt{2 {h}_{0} L cot θ} ) {sin}^{2} ( θ )} cos ( \frac{2 π t}{T} ) , θ > {θ}_{0}\end{matrix} \qquad Equation 2$$

In the example embodiment, Equation 1 may be used to calculate a critical angle when a fluid-free surface comes into contact with an edge of the MPS bottom (16.7 degrees). Equation 2 may be used to calculate a maximum shear stress. Viscosity may be a critical component of shear stress and a published value of DMEM's viscosity of 0.731 mPa s−1 is used to perform the calculation. The shear calculation may use several assumptions, including a no-slip boundary condition at a bottom of the dish, a zero-velocity gradient at a fluid-free surface, flow mainly from gravity, and that an effect from gravity is much greater than viscous effects. In addition, centrifugal force is neglected due to slow angular speed, velocity is assumed to be normal to the dish bottom, and a pressure gradient along the fluid depth is ignored. In the example embodiment, a calculated shear value is assumed to be similar across all experiments utilizing the aforementioned properties of the rocker.

FIGS. 8A-8C illustrate gravity-driven flow across epithelial cells according to an example embodiment. FIG. 8A illustrates a microfluidic system 800 on a rocker 854-1 configured to hold the microfluidic system 800 at a first orientation. At the first orientation, a media 812-1 is held by gravity within a first reservoir of two media reservoirs, e.g. 810. The media reservoirs are fluidically coupled to a channel 808. A port 816 is configured to enable access to a second channel (not shown, but similar to the second channel 334 described herein with reference to FIG. 3). FIG. 8B illustrates the microfluidic system 800 after at least a portion of a media 812-2 has started to flow from a first reservoir of the two media reservoirs, e.g., 810, into the channel 808. For example, FIG. 8B may illustrate the microfluidic system 800 after a rocker, e.g., the rocker 854-1, has started to change orientations. FIG. illustrates the microfluidic system 800 on a rocker 854-2 configured to hold the microfluidic system 800 at a second orientation. At the second orientation, gravity may drive flow of the media 812-3 from a first reservoir of the at least two reservoirs, e.g. 810, (the left reservoir) into a second reservoir of the at least two reservoirs, e.g., 810 (the right reservoir). The media 812-3 flows through the channel 808 fluidically coupled to the at least two media reservoirs, e.g., 810. Chambers configured to hold cell cultures may be fluidically coupled to the channel 808 and gravity-driven flow may enable flow of the media 812-3 with respect to the cell cultures, which may exert shear stress on the cell cultures.

Optimization of an experimental timeframe may be completed to support enteric neuron maturation and extension in the 3D environment of the MPS as well as epithelial monolayer differentiation.

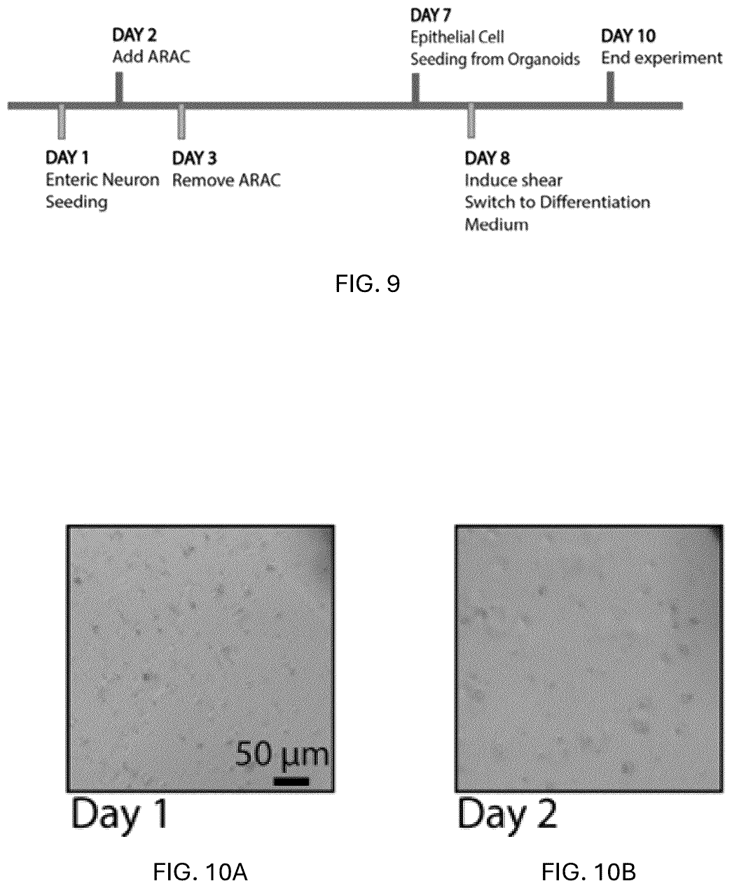

FIG. 9 illustrates an experimental timeframe according to example embodiment of a microphysiological system. Neurons are allowed to mature for 7 days within the MPS before epithelial seeding to allow adequate time for neurite extension throughout a gel layer of the device. Epithelial cells are added to the MPS on the 7th day of neuronal culture. Neurons and epithelium are co-cultured for 3 days before endpoint analysis. Epithelial cultures are halted after 3 days in co-culture due to the reported high turnover rate of small intestinal epithelial cells, which may limit the time frame of these experiments without a source for intestinal renewal. Epithelial shedding in vitro may be similar to anoikis in the gut where shedding of intestinal lining occurs after 2-6 days. Twenty-four hours after epithelial seeding, the MPS is moved from static culture conditions to a laboratory rocker to supply shear across an epithelial monolayer.

In an example embodiment of an experiment described herein with reference to FIG. 9, primary neonatal rat enteric neurons may be cultured in three dimensions in an MPS. Epithelial monolayers may be seeded from rat neonatal duodenal organoids. Using neonatal intestines may enable observation of early epithelial development and differentiation. Most organoids maintained a spheroid morphology with limited budding up to seven days before passage. Seven days post neuron seeding, epithelial cells from organoids were seeded onto the top membrane layer of the chip to form a monolayer. Epithelial cells received a differentiation medium and were placed on the rocker to supply shear 24 h after being seeded on the MPS. On the tenth day from initial neuron loading, experiments were ended. Cells used in this model were sourced from neonatal rat cells, which may be potentially less indicative of mature, human adult tissue.

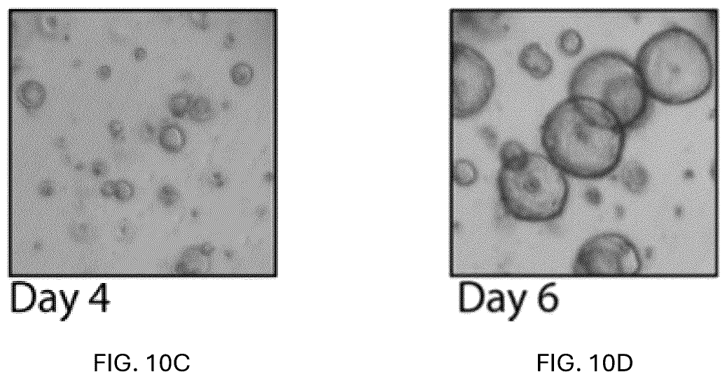

FIGS. 10A-10D illustrates brightfield images of epithelial crypt cells isolated from a duodenum and expanded as intestinal organoids in a 3D culture environment prior to use in experiments, according to an example embodiment. The figures illustrate the epithelial crypt cells at day 1 (FIG. 10A), day 2 (FIG. 10B), day 4 (FIG. 10C), and day 6 (FIG. 10D) of culture and indicate formation of intestinal organoids in a 3-D culture environment by day 6 before use in experiments.

Epithelial Cell Adhesion and Enteric Neurite Extensions Occur in an Interfacing Co-Culture MPS

Example embodiments of the high throughput chip design may be utilized for all cell culture experiments. Healthy cell morphologies with adherent epithelium and spindly neurons, were confirmed using an inverted light microscope prior to running endpoint experiments. The glass bottom and transparent membranes within the chip may allow for clear imaging through the different layers of the coculture.

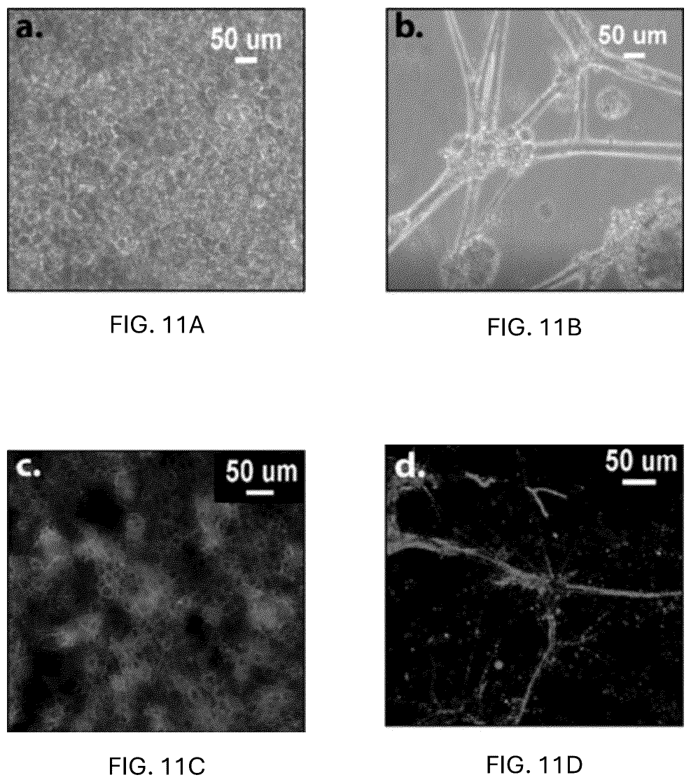

FIGS. 11A and 11B illustrate brightfield images of an epithelial monolayer and a plane of a gel containing enteric neurons, respectively, in a microphysiological system before fixation, according to an example embodiment. FIG. 11A illustrates epithelial cells developing a cobblestone morphology adhering to an ECM-coated membrane, which may be unlike budding spheres formed by epithelial cells while expanding (for example, the intestinal organoids described herein with reference to FIG. 10D).

FIGS. 11C and 11D illustrate immunostained layers of an epithelial monolayer and a plane of a gel containing enteric neurons, respectively in a microphysiological system, according to an example embodiment. Cells of the epithelial monolayer may be stained using antibodies targeting tight junction protein Zonula Occludens-1 (ZO-1) and enteric neurons may be stained using antibodies targeting Beta III Tubulin.

As illustrated in FIGS. 11B and 11D, the enteric neurons may develop neurite extensions, forming ganglionic-like morphology confirmed with imaging. FIG. 11 D may also highlight a presence of enteric neurons in the co-culture device based on features of the neuron structural marker, beta III tubulin, within a single plane of the of the neuronal compartment of an MPS. FIGS. 11A and 11C illustrate mature epithelial cells may form a characteristic cobblestone pattern and indicate expression of ZO1 at borders between the cells. However, cell surfaces did not maintain a uniform matured epithelium, which may be indicated by some areas of cytoplasmic ZO1 staining. The lack of a uniform matured epithelium may be due to enterocytes' fast cell turnover period within a duodenum in view of 3-day timeframe for experiments.

Embodiments of microphysiological systems presently described may provide a unique feature wherein a 3-D neuronal chamber is directly underneath, and proximal to, an epithelial monolayer. Transwell systems may not support direct interfacing between cell populations, such as the interfacing achieved by MPSs described herein. In some embodiments of an MPS, a porous membrane may include membrane pore sizes of around 1 μm. The membrane pore size may be large enough to support neurite extension through a gel layer and the membrane pores. Such an architecture in an MPS may provide a potential of direct contact between neurons and epithelium, which may facilitate both paracrine and synaptic communication.

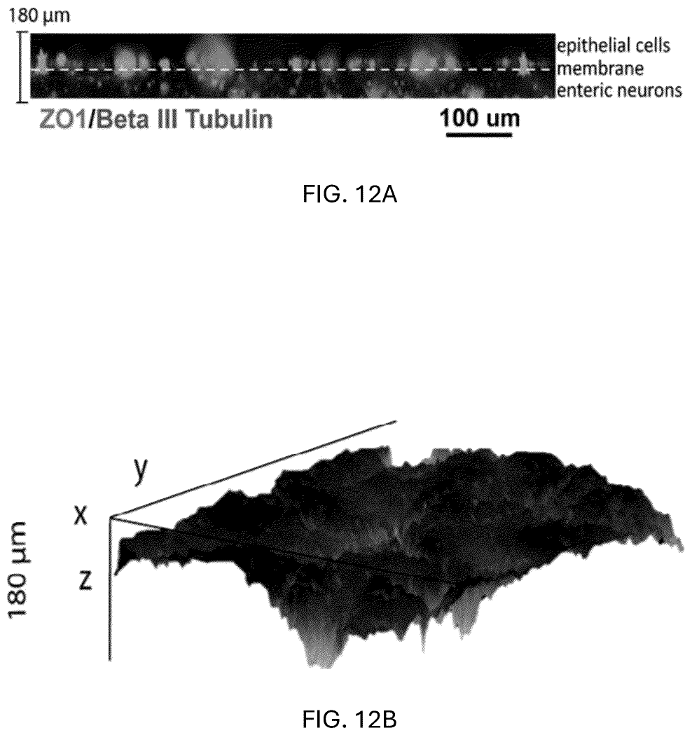

FIG. 12A illustrates an orthogonal maximum intensity projection showing layers of a culture, including beta III tubulin identifying neurons and ZO-1 identifying epithelial cells, according to an example embodiment. The orthogonal projection of a z-stack indicate a distinct neuronal layer (purple, beta III tubulin) with some potential extensions through a membrane up to a top layer of epithelial cells expressing ZO1 in green.

FIG. 12B illustrates a 3-D surface plot showing proximity and depth of different cell types in an example embodiment of a culture system. The 3-D surface plot rendering of ZO1 and beta III tubulin expression shows intensities throughout the z-stack of a given example co-culture in an MPS. The surface plot displays different depths that cell populations reside in as well as proximity of the neurons to the epithelium.

Enteric Neuron Cultures Represent a Heterogeneous Population with Cholinergic and VIPergic Subtypes Potentially Modulating the Epithelial Function

Enteric neurons may interact closely with the gut epithelium in vivo but are traditionally absent from in vitro gut models. Similar to the central nervous system, enteric neurons may be composed of several subtypes responsible for different functions within the intestine, including modulating epithelial barrier formation and differentiation.

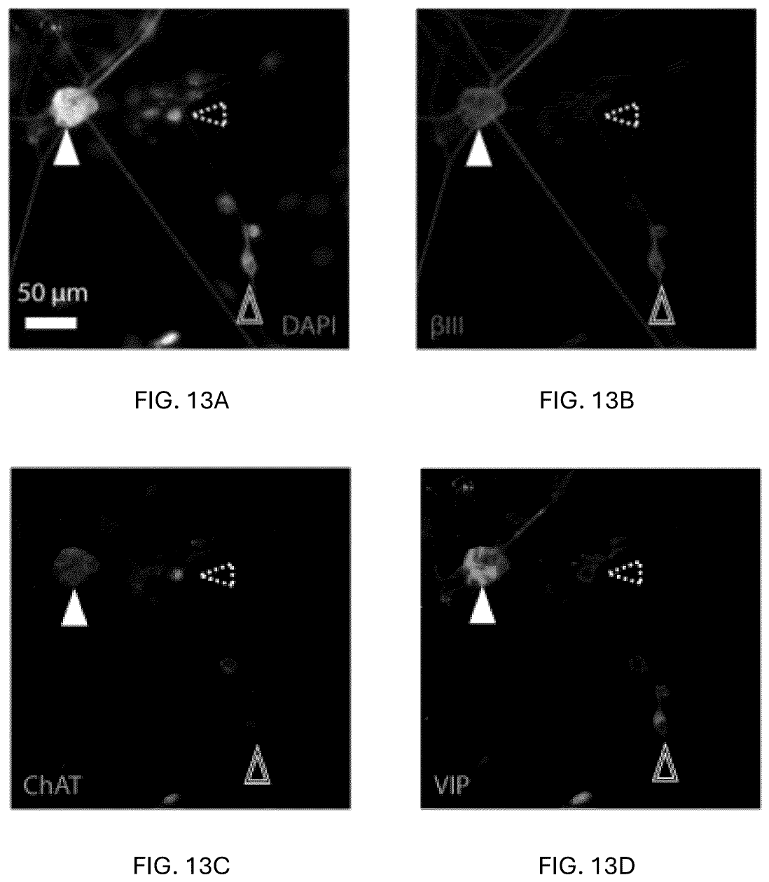

FIGS. 13A-D illustrate immunocytochemistry images of cultures of rat neonatal enteric neurons under standard culture conditions according to an example embodiment. Furthermore, specific subtypes of neurons may be identified using immunostaining. Specific immunostaining agents may target cell nuclei (4′,6-diamidino-2-pehnylindole, DAPI; FIG. 13A), neurons (beta III tubulin; FIG. 13B), cholinergic neurons (choline acetyltransferase, ChAT; FIG. 13C), and vasoactive intestinal peptide (VIP)-ergic (VIP; FIG. 13D) neurons.

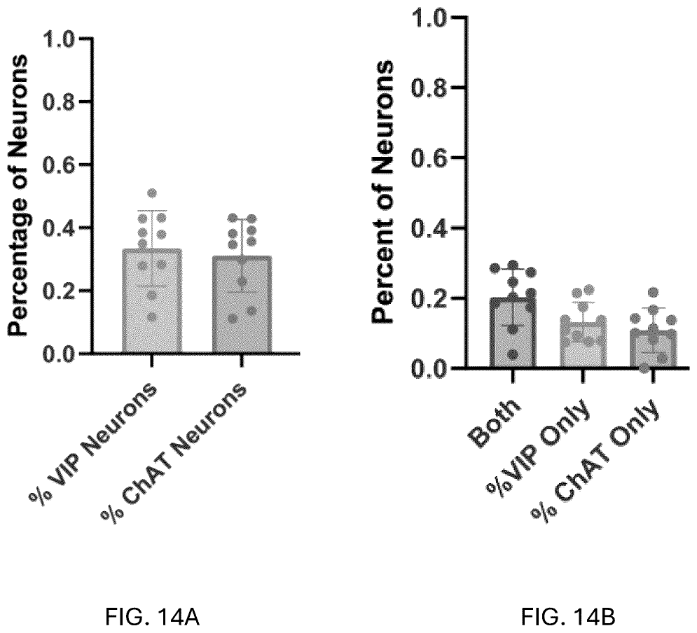

FIGS. 14A and 14B illustrate plots of a percentage of neurons expressing vasoactive intestinal peptide or choline acetyltransferase in cultures similar to those of FIGS. 13A-D. Analysis of immunocytochemistry images indicate a heterogeneous population of neurons with an average of 31.1% (±10.2%) expressing ChAT and 33.4% (±10.3%) expressing VIP, as illustrated in FIG. 14A. Neuronal expression of ChAT and VIP may be further broken down to quantify ratios of ChAT only and VIP only expressing neurons, with 13.2% (±5.55%) expressing VIP only, 10.8% (±6.56%) expressing ChAT only, and 20.3% (±7.82%) expressing both markers.

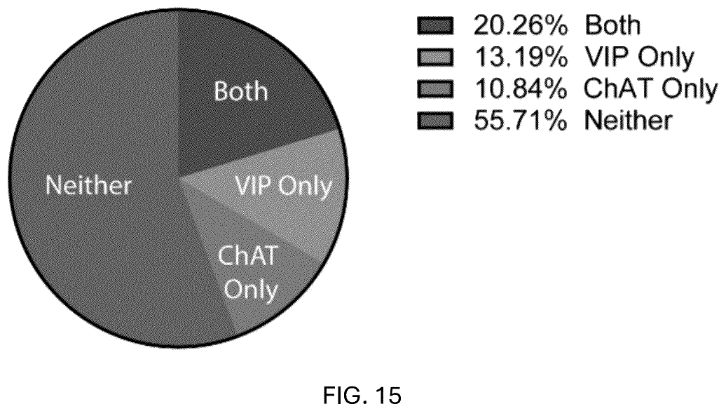

FIG. 15 illustrates a plot of a percentage of neurons expressing vasoactive intestinal peptide or choline acetyltransferase out of a total number of cells in the cultures of FIGS. 13A-D. The plot of FIG. 15 further expands upon the plots of FIGS. 14A and 14B, indicating that 55.7% (+13.2%) neurons within the cultures express neither ChAT and VIP.

The findings reported in FIGS. 14A, 14B, and 15 may differ slightly from prior literature estimating that the human small intestine contains 5-15% VIPergic and 50-70% cholinergic neurons. Such differences may be a result of source organism and maturity, as experiments presented herein utilize neonatal rat tissue. The diversity of enteric neuron subtypes remains understudied in the literature. In the experiment described herein with respect to FIGS. 14A, 14B, and 15, the neuron cultures included a majority VIPergic population, which may contribute to a stronger barrier in epithelial cells within example embodiments of a co-culture system. However, several other neuronal subtypes have yet to be explored in the embodiments and may contribute to epithelial interactions.

### Co-Cultured Epithelial Cells and Enteric Neurons had More Robust Barrier Properties Compared to Monocultured Epithelium

In some embodiments of a 12-unit MPS system, the MPS may be designed to support real-time on-chip assays for assessing live epithelial barrier properties, including TEER and apparent permeability.

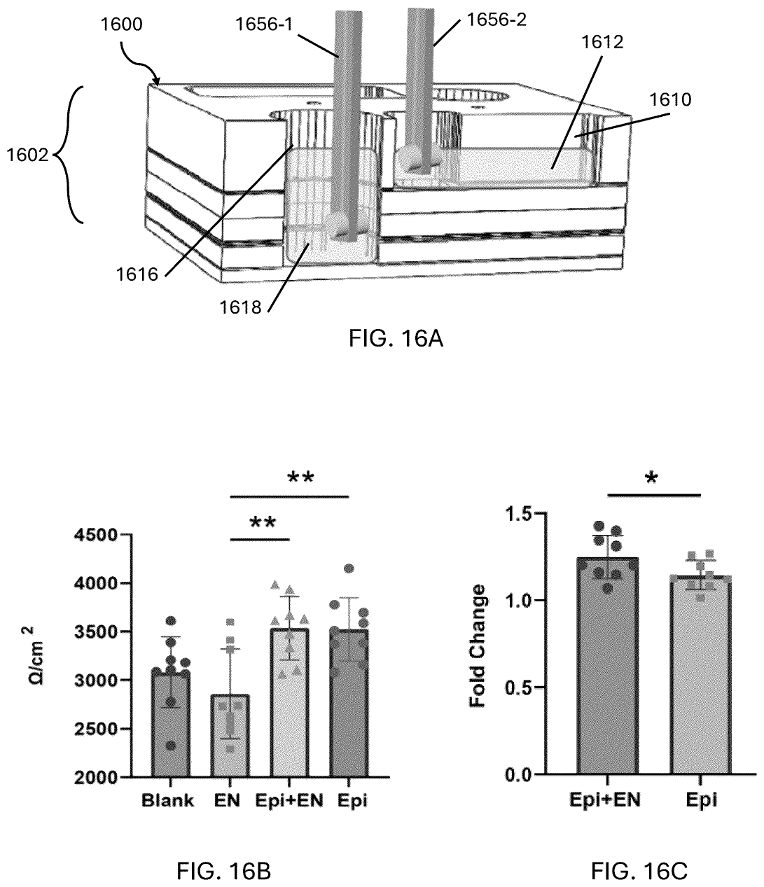

FIG. 16A illustrates a schematic of a TEER assay in an example embodiment of a microfluidic device 1600. TEER may be measured using an epithelial volt ohm device with chopstick electrodes 1656-1, 1656-2, and measurements may be conducted on day 10 of an experiment similar to that described herein with reference to FIG. 9. The microfluidic device 1600 may be similar to the microfluidic device 100 described herein with reference to FIG. 1. A plurality of layers 1602 of the microfluidic device 1600 includes a first channel layer configured to define a first channel (not shown) and a second channel layer configured to define a second channel (not shown). The plurality of layers is further configured to define one or more ports, including a port 1616 fluidically coupled to the second channel and configured to enable dispensing of a basal media 1618 into the second channel. A reservoir layer is configured to define at least one media reservoir, e.g., 1610, fluidically coupled to the first channel through another port (not shown) of the one or more ports. The at least one media reservoir, e.g., 1610, is configured to enable dispensing of a luminal media 1612 into the first channel through the another port. Design of media inlets of the microfluidic device 1600, i.e., the port 1616 and the at least one media reservoir, e.g., 1610, is sufficiently large to support insertion of electrodes into the chip, similar to a standard Transwell system.

FIGS. 16B and 16C illustrate results of a transepithelial electrical resistance assay, which may be similar to the TEER assay schematically illustrated in FIG. 16A, according to an example embodiment. The results from FIGS. 16B and 16C may be collected from an example embodiment of a 12-unit chip and include three samples (n=3) with three measurements per sample (m=3), and significance is indicated by * for p<0.05 and ** for p<0.01. TEER values for respective monoculture controls, epithelial and ENS co-cultures, and blank controls are determined and compared. As shown in FIG. 16B, TEER values for enteric neuron (EN) only cultures may be measured to use as an appropriate control against co-cultured groups to account for changes neurons may make to bulk resistance values. Enteric neuron-only control cultures exhibited lower resistances than blank gel control (2860±462.6 Ω cm−2 versus 3083±365.3 Ω cm−2), which may be due to the glia degrading and migrate through the gel matrix, increasing porosity and resistance to passive ion transport. Although glia may be reduced using cytosine arabinoside (ARaC), a fraction of glial cells may persist and result in gel remodeling, which may occur through protease secretion. FIG. 16C illustrates fold-change calculated for each group, transforming the data by the appropriate control condition. The epithelial-only TEER values were transformed using resistance measured from the blank gel, and co-culture data was transformed using measured enteric neuron resistance values. This fold change calculation may control for resistance changes due to the neurons and may allow for assessment of absolute TEER of the epithelial monolayers. The plot of FIG. 16C indicates a significantly higher fold change in the co-cultured group at 1.25±0.124 average fold, compared to the epithelium only at 1.14±0.0835-fold (p=0.049).

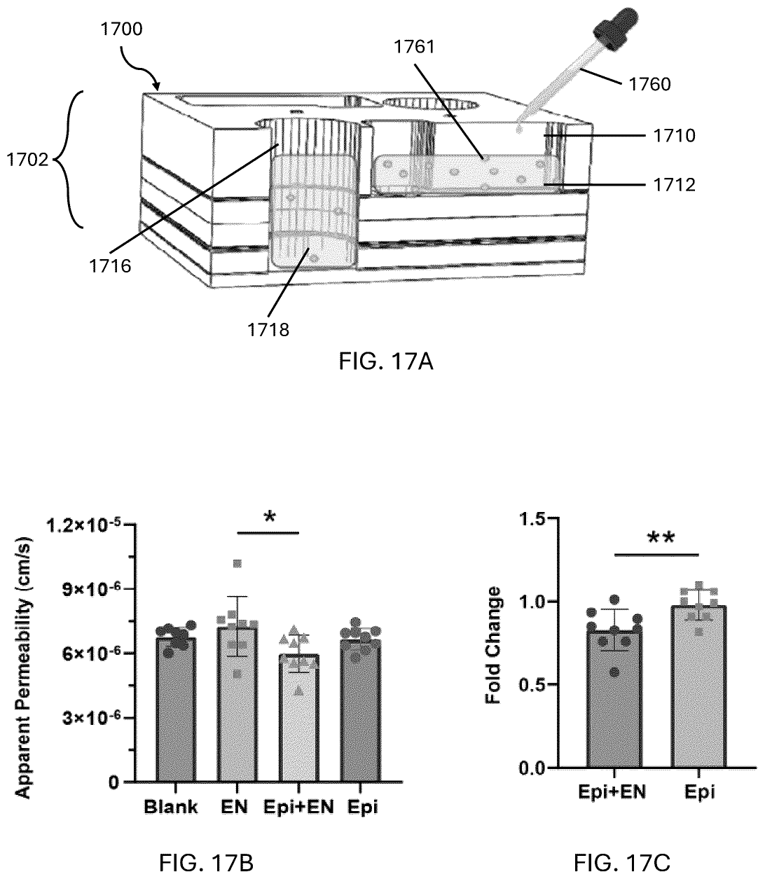

FIG. 17A illustrates a schematic of an apparent permeability assay in an example embodiment of a microfluidic device 1700. The microfluidic device 1700 may be similar to the microfluidic device 1600 described herein with respect to FIG. 16 and similar components are labeled using like reference numbers but incremented by 100. The microfluidic device includes a plurality of layers 1702 defining a media reservoir 1712 and a port 1716. The media reservoir 1710 is fluidically coupled to a first channel and a first chamber for holding a first cell culture (not shown) and the port 1716 is fluidically coupled to a second channel and a second chamber for holding a second cell culture (not shown). A luminal media 1712 is added to the media reservoir 1710 and a basal media 1718 is added to the port 1716. Fluorescent molecules, e.g., 1761, may be added to the media luminal media 1712 using a dropper 1760 and allowed to diffuse through an epithelial layer in the first chamber. Fluorescent molecules, e.g., 1761, diffusing through the epithelial layer may diffuse through the second chamber and second channel, flowing into the basal media 1718. The fluorescent molecules, e.g., 1761, in the basal media 1718 may be assayed to determine membrane permeability. Fluorescent molecule diffusion across an epithelium may be measured in parallel to TEER on day 10. Examples of a fluorescent molecule to measure apparently permeability from between an apical and basal compartment, e.g., the media reservoir and the second channel, may include Lucifer yellow.

FIGS. 17B and 17C illustrate results of an apparent permeability assay, which may be similar to the apparently permeability assay illustrated in FIG. 17A, according to an example embodiment. The results reported in FIGS. 17B and 17C include three samples (n=3) with three measurements per sample (m=3), and significance is indicated by * for p<0.05 and ** for p<0.01. FIG. 17B shows raw apparent permeability values for the assay. Example embodiments of an MPS used for the assay includes thicknesses with a high ratio of media, membranes, and gel compared to epithelial monolayer, which may result in the insignificant changes in the raw apparent permeability values between cocultured and monocultured groups. FIG. 17C illustrates fold change of cells cultures, and the fold change may be calculated using a method similar to that used for TEER measurements, subtracting the permeability values of the two control groups (blank and neuron only) from the epithelial monoculture and co-cultured groups, respectively. Significantly lower permeability fold change of 0.83±0.125 may be observed for the epithelial and enteric neuron co-culture compared to the epithelial-only monolayer of 0.98±0.0904 fold (p=0.0093). The fold change findings align with the TEER results and may indicate that enteric neurons help boost epithelial integrity. The experiments and findings described herein with reference to FIGS. 16A-17C may also highlight the ability to perform multiple high-throughput assays on-chip across several experimental groups.

In addition to permeability and TEER assays, coverage of an entire area of an MPS by an epithelial layer may be characterized using phalloidin-stained epithelial cells at a similar time point as the assessment of barrier function, e.g., on day 10.

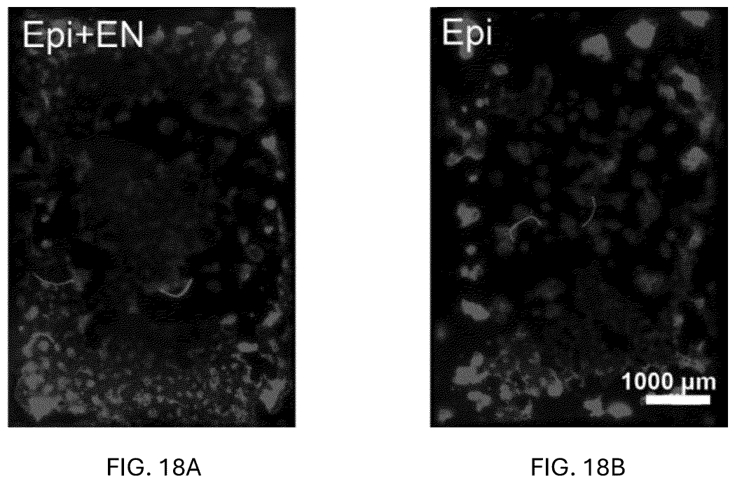

FIGS. 18A and 18B illustrate microscopy images of monolayers in example embodiments of a microphysiological device. Edges of the tiled region are brighter due to reflection of light from acrylic. FIG. 18A illustrates an image of a monolayer of an MPS with a co-culture of epithelial cells and enteric neurons and FIG. 18B illustrates an image of a monolayer of an MPS with only epithelial cells.

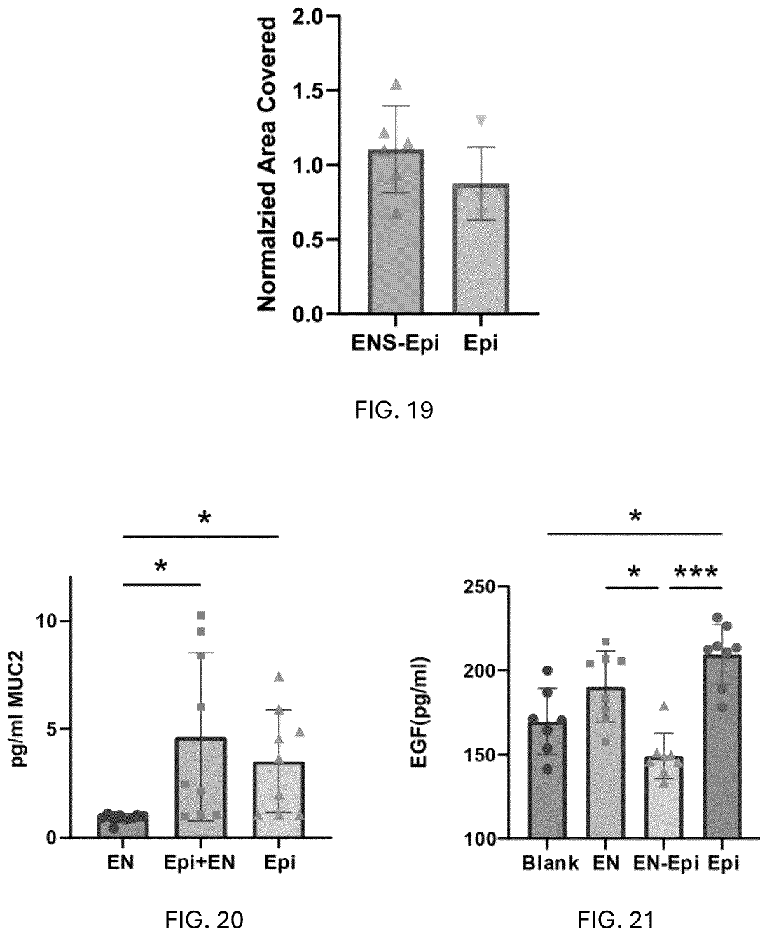

FIG. 19 illustrates a plot of normalized area covered for microscopy images similar to the microscopy images of FIGS. 18A and 18B according to an example embodiment. An area covered by cells for each sample may be normalized by an average of all sample areas, combining mono- and co-culture, of a full technical replicate (n) to remove some interexperimental variability. Epithelial-only and co-cultured groups were compared, showing a higher normalized area coverage of 1.1048±0.291 for the co-cultured group with enteric neurons present, compared to epithelial cells, with only with a mean normalized coverage of 0.8742±0.243. Improved monolayer coverage may support findings of the TEER and Lucifer yellow permeability assays and the hypothesis that enteric neurons promote an improved epithelium barrier function in an in vitro system. Enteric neurons have been found to increase cell proliferation and barrier integrity of the epithelium in previous experiments through tight junction and mucus formation. These past findings may pose a question of if the microenvironments described herein have detectable mucus concentrations.

### Co-Cultures of Enteric Neurons and Epithelium Produce a Trending Increase in Mucus Compared to Epithelial Only Cultures

FIG. 20 illustrates a plot of concentrations of mucin 2 (Muc2) in cell cultures according to an example embodiment. A Muc2 enzyme-linked immunosorbent assay (ELISA) may performed with apical supernatants (e.g., the luminal media 112 described herein with reference to FIG. 1) collected from microphysiological systems including epithelial cells cultured with and without neurons, as well as neuron only and blank media controls. Both the epithelial monoculture (2.43±1.50 pg mL−1, p=0.048) and co-culture (3.25±2.46 pg mL−1, p=0.026) had significantly higher Muc2 concentrations than the enteric neuron monoculture (0.59±0.0796 pg mL−1), supporting mucus production in our MPS microenvironment. Although co-cultures produced 33% more Muc2 than the monocultured epithelium, these findings were not statistically significant and may not support a conclusion that barrier differences seen with neurons present were due to increases in mucus in the culture.

Co-Cultures of Enteric Neurons and Epithelium Contain Significantly Less EGF than their Monoculture Counterparts

FIG. 21 illustrates a plot of concentrations of epidermal growth factor (EGF) in cell cultures according to an example embodiment. EGF may be an important factor involved in regulating epithelial barrier proliferation and differentiation. An ELISA may be performed to measure overall EGF concentration found in mono- and coculture media supernatants due a potential known impact of EGF on epithelial barrier function. An epithelial media that includes 5 ng mL−1 of EGF may be added that either degrades or is consumed over a course of an experiment similar to the experiment described herein with reference to FIG. 9. The blank still contained 169.7±19.7 pg mL−1 which is significantly less than the monocultured epithelium (209.6±17.79 pg mL−1, p=0.0013), which may suggest that epithelium alone (the monocultured epithelium) may produce EGF into the supernatant. Co-cultured epithelium and ENS contained significantly less EGF than either monoculture condition. The mean EGF concentration of the co-cultured sample was 149.3±13.5 pg mL−1, compared to 190.3±21.05 and 209.6±17.79 pg mL−1 for neuron only and epithelium only, respectively (p=0.006 and p<0.0001).

The findings of the EGF assay indicate lower levels of EGF in co-cultured epithelium supernatant, which may represent a decrease in EGF production when neurons are present in a co-culture. The epithelium secretes EGF from enterocytes and Paneth cells, while the proliferative crypt cells have receptors for the EGF. A lower EGF concentration in the supernatants may be due to fewer enterocytes or Paneth cells in epithelia cultured with neurons, and potentially more intestinal epithelium stem cells. Further characterization of compositions of epithelia under mono- and co-culture conditions may further break down the source and consumption of EGF in our cultures. However, a major takeaway may be that the enteric nervous system influences growth factor levels present in intestinal epithelia and GI models lacking innervation may be missing a vital component to gut renewal. The profound difference in EGF in the basal supernatant may emphasize the importance and great differences present in heterogeneous populations in in vitro models.

Wnt and Inflammatory Genes were Varied Between the Monoculture and Co-Cultured Epithelium

In experiments performed using example embodiments of a high throughput microphysiological system, epithelial cells in monoculture and co-culture conditions may be lifted from the MPS with TryplE. RNA sequencing was performed across two pooled samples for monocultured and co-cultured epithelium with 3 pooled monolayers per sample. Two samples of freshly isolated duodenal crypt tissue were also sequenced for comparison.

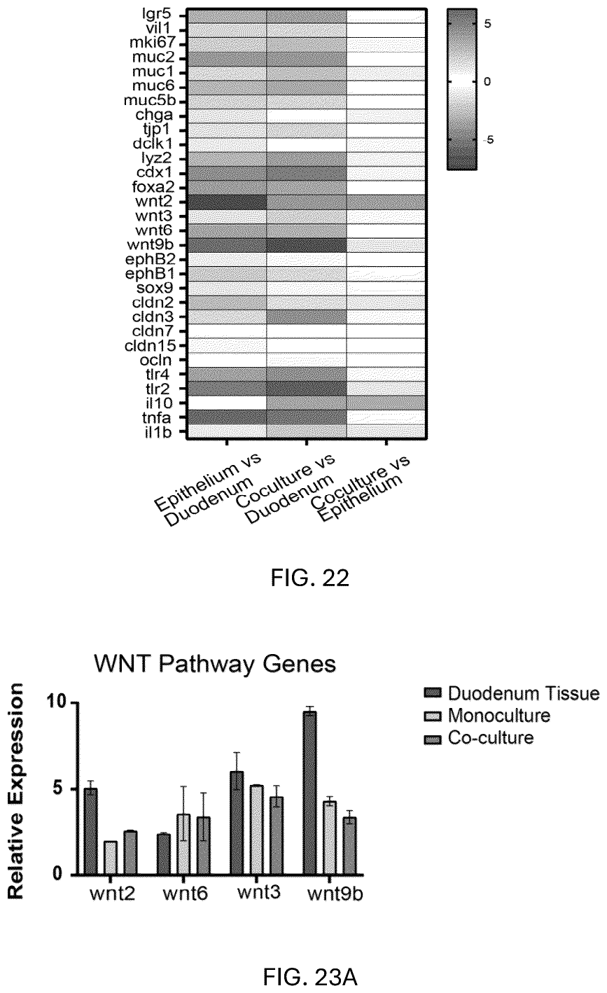

FIG. 22 illustrates differential expression (log 2-fold change) for epithelial genes of interest compared across the three experimental groups (epithelium monoculture in MPS, epithelium and enteric neuron co-culture in MPS, and duodenal crypt groups). Relative expression levels for genes of interest were divided into functional categories (Wnt pathway, proliferation/barrier function, mucin production, and inflammatory cytokines) and reported in Table 1.

TABLE 1
Relative expression (Log₂) of RNA sequencing of genes

| | | | |
|---|---|---|---|
| | Duodenum | Monocultured | Co-cultured |
| Gene ID | Tissue | Epithelium | Epithelium |

WNT Genes of Interest

| | | | | | | |
|---|---|---|---|---|---|---|
| wnt2 | 5.47 | 4.68 | 2.00 | 2.00 | 2.58 | 2.63 |
| wnt6 | 2.49 | 2.36 | 2.00 | 5.16 | 2.00 | 4.79 |
| wnt3 | 4.97 | 7.13 | 5.25 | 5.22 | 3.98 | 5.20 |
| wnt9b | 9.78 | 9.27 | 4.03 | 4.59 | 2.98 | 3.77 |

Proliferation and Tight Junction Formation Genes Of Interest

| | | | | | | |
|---|---|---|---|---|---|---|
| lgr5 | 6.58 | 10.86 | 6.93 | 6.78 | 6.67 | 6.07 |
| mki67 | 12.35 | 13.59 | 10.53 | 11.57 | 10.12 | 10.86 |
| vil1 | 15.07 | 14.78 | 13.26 | 13.10 | 12.99 | 13.44 |
| tjp1 | 12.61 | 12.42 | 13.47 | 14.22 | 13.95 | 14.66 |
| ocln | 11.58 | 12.06 | 11.91 | 11.94 | 12.05 | 12.65 |
| cldn1 | 7.14 | 6.24 | 9.76 | 11.60 | 10.35 | 12.80 |
| cldn3 | 7.32 | 9.19 | 4.46 | 11.20 | 4.43 | 11.36 |

Mucin Genes of Interest

| | | | | | | |
|---|---|---|---|---|---|---|
| muc2 | 15.83 | 15.90 | 12.67 | 10.64 | 12.49 | 10.94 |
| muc6 | 8.42 | 10.98 | 6.99 | 7.74 | 6.64 | 7.22 |
| muc1 | 7.72 | 9.49 | 11.05 | 9.63 | 12.21 | 8.79 |
| muc13 | 8.82 | 9.69 | 5.63 | 10.35 | 6.14 | 10.59 |
| muc5b | 11.86 | 13.09 | 11.78 | 4.44 | 11.85 | 5.36 |
| muc15 | 3.38 | 2.88 | 4.69 | 3.89 | 3.98 | 3.51 |

Inflammatory Genes of Interest

| | | | | | | |
|---|---|---|---|---|---|---|
| il18 | 10.60 | 10.28 | 4.11 | 6.98 | 4.78 | 7.88 |
| il10 | 3.14 | 2.51 | 2.00 | 3.55 | 2.98 | 6.20 |
| il1b | 6.43 | 4.63 | 5.46 | 7.41 | 5.61 | 8.75 |
| tnfa | 3.38 | 2.35 | 7.31 | 7.78 | 7.40 | 6.66 |
| tlr4 | 4.21 | 3.26 | 6.30 | 7.45 | 6.52 | 8.16 |

Neuronal Genes of Interest

| | | | | | | |
|---|---|---|---|---|---|---|
| ncam2 | 4.81 | 4.38 | 2.00 | 2.00 | 2.00 | 4.83 |
| st6galnac5 | 4.33 | 4.32 | 2.00 | 2.00 | 2.58 | 3.84 |
| slc2a | 7.55 | 6.70 | 2.00 | 2.00 | 2.00 | 2.94 |
| grik2 | 2.00 | 2.64 | 2.00 | 2.47 | 2.00 | 4.29 |
| tac1 | 5.18 | 6.01 | 3.65 | 6.19 | 5.29 | 9.23 |

Acetylcholine and VIP Receptor Genes of Interest

| | | | | | | |
|---|---|---|---|---|---|---|
| chrm3 | 8.84 | 9.53 | 9.19 | 9.84 | 9.00 | 9.74 |
| chrna10 | 2.49 | 4.78 | 2.00 | 3.64 | 2.00 | 2.25 |
| vipr2 | 6.28 | 8.47 | 3.85 | 3.64 | 2.57 | 3.42 |

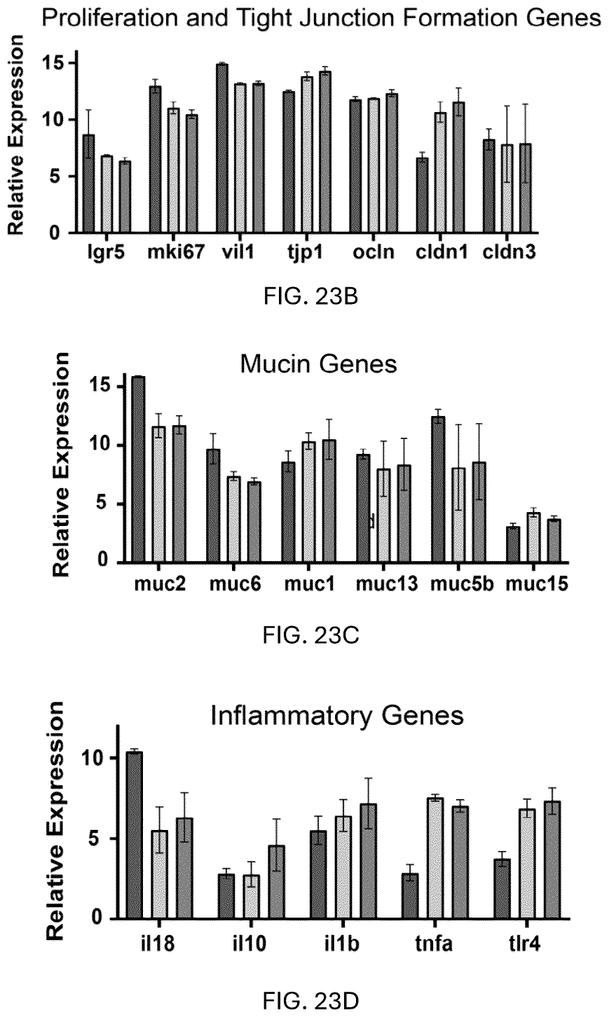

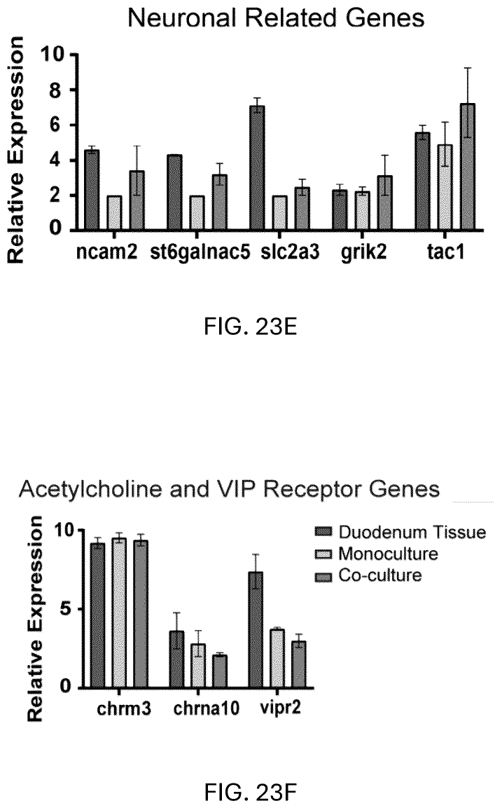

FIGS. 23A-F illustrate plots of relative gene expression of the different categories of genes for the experimental groups of FIG. 22. The legend found in FIG. 23A may be applied to all of the FIGS. 23A-F.

FIG. 23A illustrates a plot of relative gene expression for the Wnt pathway, including Wnt2, Wnt3, Wnt6, and Wnt9B. Pathways related to Paneth cells (Wnt3, Wnt6, Wnt9B) demonstrate similar expression between the co-culture and monoculture. However, the co-culture group had a higher differential expression, as shown in FIG. 22, of the Wnt2 gene compared to the epithelium monoculture. Wnt2 has been found to inhibit inflammation and apoptosis in response to bacterial infection in vivo, and ablating and knocking down Wnt2 function may decrease proliferation of colorectal cancer. Previous literature may indicate that Wnt2 expression might be inhibiting apoptosis and increasing proliferation in our co-culture samples

FIG. 23B illustrates a plot of relative gene expression of genes associated with tight junction formation and proliferation. Proliferation genes (Lgr5, Mki67) were slightly higher for monoculture samples than co-cultured (0.48- and 0.64-fold, respectively), but expression of both proliferation genes for monoculture and co-culture samples were lower than that found in duodenal tissue. In general, lower or equivalent expression of tight junction protein genes (Tjp1, Vil1, Ocln, Cldn3) is shown in monocultured and co-cultured MPSs as compared to duodenal cells. However, Claudin 1 (Cldn1) demonstrated higher expression in the MPS samples than the tissue, with the co-culture exhibiting the highest expression. A higher expression of Cldn1 may indicate a population of more goblet cells and/or Paneth cells.

FIG. 23C illustrates a plot of relative gene expression of genes associated with mucin production. A majority of mucin genes (Muc2, Muc6, Muc5b) investigated were expressed in lower levels in MPS groups compared to duodenal tissue. On the other hand, Muc1 expression was found to be higher in the monoculture and co-culture MPS groups. Compositions of media used in these experiments may aim to maintain a monolayer for longer by enriching for proliferative cell types (including valproic acid and CHIR99021) and inhibiting apoptosis (Y-27632, ROCK inhibitor), which may decrease the overall quantity of mucus producing goblet cells in these cultures as compared to the freshly isolated tissue. However, mucin producing genes were expressed similarly across the MPS samples, not matching the trend seen in the Muc2 production ELISA experiment described herein with reference to FIG. 20.

FIG. 23D illustrates a plot of relative gene expression of genes associated with inflammation. Inflammatory genes were investigated due to their potential to influence overall epithelial health. Generally, MPS samples (monoculture and co-culture) showed higher levels of inflammatory markers like interleukins (IL-10, IL-1b) and toll like receptor genes (Tlr4, Tlr2) compared to the duodenal tissue. Tnfa and Tlr4 had higher expression in the MPS samples compared to tissue. However, IL-18 was lower in the MPS samples compared to duodenal crypt tissue. The co-culture and monoculture samples were similar across most inflammatory genes of interest, except for IL-10 which was 3.24-fold higher in the co-cultured group. IL-10 may have a number of effects on the intestinal epithelium including supporting differentiation into goblet and Paneth cells, supporting stem cell proliferation, promoting cell repair, and preventing apoptosis. Epithelial cells that interacted with neurons were found to have higher expression levels for the anti-inflammatory cytokine, IL-10, which may correspond with previous data for non-contacting co-cultures in a Transwell system.

The experiments using example embodiments of MPSs described herein may suggest that enteric neurons promote expression of IL-10 in duodenal epithelium. Combined trends of enriched Wnt, Cldn1, and IL-10 genes may further suggest that co-cultured samples contain more Paneth cells or goblet cells than the monocultured epithelium. However, the Paneth cell (Lyz2) and goblet cell (Muc group of genes) are not as profoundly enriched and may not support previously stated conclusion.

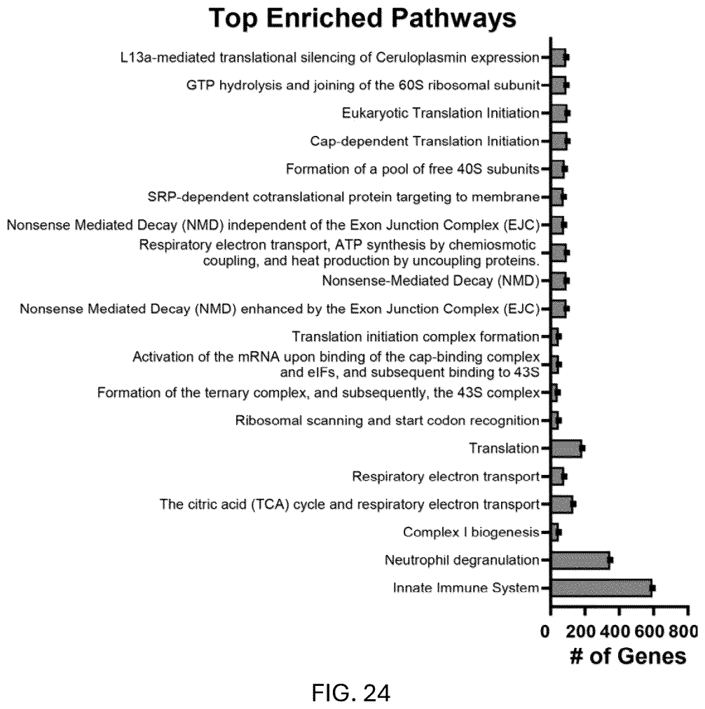

FIG. 24 illustrates a plot of a top 20 pathways enriched by presence of neurons in in vitro co-cultures, according to an example embodiment. The 20 pathways are reported as normalized enrichment scores (NES). Interestingly, innate immunity and neutrophil degranulation experienced the highest number of gene hits. The pathway with the highest enrichment score was L13a-mediated translational silencing of ceruloplasmin expression, which may also contribute to immune function.

TABLE 2
Enriched reaction pathways for co-cultured
versus monocultured epithelial cells

| | | Normalized |
|---|---|---|
| | | Enrichment |
| | # of | Score |
| Pathway | Genes | (NES) |
| L13a-mediated translational silencing of | 91 | 2.7521 |
| Ceruloplasmin expression | | |
| GTP hydrolysis and joining of the 60S ribosomal | 92 | 2.7313 |
| subunit | | |
| Eukaryotic Translation Initiation | 98 | 2.7005 |
| Cap-dependent Translation Initiation | 98 | 2.7005 |
| Formation of a pool of free 40S subunits | 82 | 2.6973 |
| SRP-dependent cotranslational protein | 76 | 2.6829 |
| targeting to membrane | | |
| Nonsense Mediated Decay (NMD) independent | 78 | 2.6539 |
| of the Exon Junction Complex (EJC) | | |
| Respiratory electron transport, ATP synthesis by | 94 | 2.562 |
| chemiosmotic coupling, and heat production by | | |
| uncoupling proteins. | | |
| Nonsense-Mediated Decay (NMD) | 93 | 2.5414 |

Epithelial RNA from Co-Cultured MPSs have Increased Neuronal Gene Expression Suggesting Presence of Extending Neurites to the Epithelial Monolayer

For some example embodiments of microphysiological systems, the top 100 differential genes were compared for the monocultured and co-cultured MPS samples (Table 7).

TABLE 3
Top 100 most differentially expressed genes in co-culture versus monocultures

| | | | EPI vs | EPI + EN |
|---|---|---|---|---|
| | | EPI + EN | Isolated | vs Isolated |
| | | vs EPI | Duodenum | Duodenum |
| | | (log2 fold | (log2 fold | (log2 fold |
| Ensemble ID | GENE ID | change) | change) | change) |
| ENSRNOG00000002126 | Ncam2 | 6.28603 | −7.00833 | −0.7223 |
| ENSRNOG00000051537 | AABR07006673.1 | 5.689542 | −3.83826 | 1.851285 |
| ENSRNOG00000058052 | AABR07060915516.1 | 5.391814 | −5.77947 | −0.38765 |
| ENSRNOG00000049676 | St6galnac5 | 5.326859 | −6.66811 | −1.34125 |
| ENSRNOG00000062950 | U13 | 5.230234 | −1.56835 | 3.661885 |
| ENSRNOG00000057709 | Snord8 | 4.847672 | −4.56659 | 0.281085 |
| ENSRNOG00000052487 | Snord73 | 4.65328 | −4.66943 | −0.01615 |
| ENSRNOG00000004302 | Pah | 4.607771 | −6.92297 | −2.31519 |
| ENSRNOG00000029191 | LOC685067 | 4.545121 | −8.69259 | −4.14747 |
| ENSRNOG00000063461 | U2 | 4.524145 | −5.73161 | −1.20747 |
| ENSRNOG00000069609 | NA | 4.455111 | −8.14492 | −3.68981 |
| ENSRNOG00000067735 | NA | 4.295336 | −5.46307 | −1.16773 |
| ENSRNOG00000055009 | U6 | 4.257838 | −4.18448 | 0.073356 |
| ENSRNOG00000006604 | Thy1 | 4.15657 | −6.32108 | −2.16451 |
| ENSRNOG00000049826 | UST4r | 4.126229 | −9.07052 | −4.94429 |
| ENSRNOG00000066167 | NA | 4.097251 | −6.15354 | −2.05629 |
| ENSRNOG00000019810 | Des | 3.979562 | −7.85227 | −3.8727 |
| ENSRNOG00000069670 | Ugt2b | 3.840931 | −5.85886 | −2.01793 |
| ENSRNOG00000008376 | Slc2a3 | 3.821664 | −9.80787 | −5.98621 |
| ENSRNOG00000068634 | Csta | 3.779134 | 0.903222 | 4.682356 |
| ENSRNOG00000051905 | Wnt2 | 3.736891 | −7.60572 | −3.86882 |
| ENSRNOG00000065655 | NA | 3.731947 | 2.167579 | 5.899526 |
| ENSRNOG00000037549 | AABR07058124.2 | 3.730844 | −5.89787 | −2.16702 |
| ENSRNOG00000068803 | U4 | 3.724455 | −2.67051 | 1.053943 |
| ENSRNOG00000068992 | NA | 3.716936 | −4.60516 | −0.88822 |
| ENSRNOG00000035617 | Mir186 | 3.658353 | −3.54045 | 0.1179 |
| ENSRNOG00000053017 | AC131411.2 | 3.634987 | −6.15361 | −2.51862 |
| ENSRNOG00000004704 | Dcstamp | 3.513132 | 3.482746 | 6.995877 |
| ENSRNOG00000006579 | Reg3g | 3.49339 | 3.984277 | 7.477668 |
| ENSRNOG00000027380 | Upk1b | 3.475379 | −3.85933 | −0.38395 |
| ENSRNOG00000004900 | Crhr1 | 3.38904 | −8.71469 | −5.32565 |
| ENSRNOG00000060046 | AABR07029664.1 | 3.383462 | −7.64743 | −4.26396 |
| ENSRNOG00000069254 | Cd209al1 | 3.383331 | −9.26983 | −5.8865 |
| ENSRNOG00000000368 | Grik2 | 3.318578 | −0.58012 | 2.738456 |
| ENSRNOG00000059833 | AABR07073270.1 | 3.306823 | −5.85931 | −2.55248 |
| ENSRNOG00000002829 | Ppbp | 3.256119 | 1.482059 | 4.738178 |
| ENSRNOG00000004647 | Il10 | 3.239131 | 0.316201 | 3.555333 |
| ENSRNOG00000057127 | Zg16b | 3.232758 | −4.96221 | −1.72945 |
| ENSRNOG00000000053 | Crp | 3.223745 | −5.86881 | −2.64507 |
| ENSRNOG00000020953 | Ms4a7 | 3.212911 | −1.26288 | 1.950036 |
| ENSRNOG00000066140 | LOC120095795 | 3.158616 | −0.59835 | 2.560269 |
| ENSRNOG00000052986 | U6 | 3.144266 | −4.3284 | −1.18413 |
| ENSRNOG00000007137 | Ly6i | 3.099948 | 0.516732 | 3.61668 |
| ENSRNOG00000019159 | Jakmip2 | 3.066593 | −0.72469 | 2.3419 |
| ENSRNOG00000021364 | LOC691311 | 3.033307 | −7.77248 | −4.73917 |
| ENSRNOG00000067502 | Tac1 | 3.023527 | −0.25245 | 2.771075 |
| ENSRNOG00000060922 | AC118127.1 | 3.019049 | −2.96095 | 0.058095 |
| ENSRNOG00000013954 | Alpl | 3.007982 | −1.24523 | 1.762756 |
| ENSRNOG00000006726 | Zfp9 | 2.98044 | −5.19878 | −2.21834 |
| ENSRNOG00000004679 | Fign | 2.97647 | −7.07128 | −4.09481 |
| ENSRNOG00000070284 | NA | −2.90871 | 1.580207 | −1.3285 |
| ENSRNOG00000071185 | NA | −2.95488 | −1.34425 | −4.29914 |
| ENSRNOG00000010047 | Ddit4l2 | −2.96854 | 1.026878 | −1.94166 |
| ENSRNOG00000070087 | NA | −2.97473 | −0.32485 | −3.29959 |
| ENSRNOG00000020293 | Chrna10 | −2.97802 | −1.55411 | −4.53213 |
| ENSRNOG00000062856 | NA | −2.98052 | −7.94207 | −10.9226 |
| ENSRNOG00000066888 | NA | −2.98194 | 5.089741 | 2.107799 |
| ENSRNOG00000017206 | Igfbp5 | −2.99046 | 0.523919 | −2.46654 |
| ENSRNOG00000021048 | AC095693.1 | −3.011 | 2.258104 | −0.75289 |
| ENSRNOG00000002501 | Ddx3y | −3.03803 | −5.46497 | −8.503 |
| ENSRNOG00000033654 | LOC501038 | −3.07014 | −5.6026 | −8.67275 |
| ENSRNOG00000051169 | Clnk | −3.07234 | −5.46025 | −8.53258 |
| ENSRNOG00000003305 | Cxcr3 | −3.07541 | −5.21188 | −8.2873 |
| ENSRNOG00000068920 | NA | −3.08279 | 2.981073 | −0.10172 |
| ENSRNOG00000002947 | Dpt | −3.09116 | −5.56348 | −8.65464 |
| ENSRNOG00000048771 | RGD1559482 | −3.2057 | −3.82111 | −7.02681 |
| ENSRNOG00000029811 | Kcne2 | −3.21567 | 1.292005 | −1.92367 |
| ENSRNOG00000064997 | NA | −3.28077 | −3.05507 | −6.33584 |
| ENSRNOG00000011754 | Myom2 | −3.30485 | 3.992981 | 0.688135 |
| ENSRNOG00000050922 | Nupr1l1 | −3.30587 | 3.148077 | −0.15779 |
| ENSRNOG00000010906 | Ccl5 | −3.44644 | −5.42981 | −8.87625 |
| ENSRNOG00000053034 | AABR07071198.1 | −3.46559 | −0.90075 | −4.36635 |
| ERCC-00017 | NA | −3.50119 | 4.303664 | 0.802473 |
| ENSRNOG00000062833 | NA | −3.50242 | −2.00071 | −5.50313 |
| ENSRNOG00000046959 | Mnx1 | −3.51529 | −2.52339 | −6.03868 |
| ENSRNOG00000069077 | NA | −3.54245 | −5.67 | −9.21245 |
| ENSRNOG00000068399 | Itgad | −3.55515 | −3.32618 | −6.88133 |
| ENSRNOG00000062675 | NA | −3.56321 | 2.69938 | −0.86383 |
| ENSRNOG00000019216 | Il12rb1 | −3.60598 | −2.59732 | −6.20331 |
| ENSRNOG00000069352 | Aqp10 | −3.61344 | −5.27251 | −8.88595 |
| ENSRNOG00000003669 | Myocd | −3.68294 | −4.31504 | −7.99798 |
| ENSRNOG00000055796 | AC103574.1 | −3.68456 | −2.85835 | −6.54291 |
| ENSRNOG00000019194 | Pitx3 | −3.78662 | 5.333423 | 1.5468 |
| ENSRNOG00000069226 | NA | −3.78912 | 5.762244 | 1.973122 |
| ENSRNOG00000019265 | Pcdh12 | −3.93002 | −3.28648 | −7.21651 |
| ENSRNOG00000011946 | Ptn | −3.95726 | −6.02338 | −9.98064 |
| ENSRNOG00000059326 | Abca9 | −3.96892 | 1.28982 | −2.6791 |
| ENSRNOG00000007310 | Klrb1b | −3.97963 | −5.78911 | −9.76874 |
| ENSRNOG00000009227 | Aplnr | −4.02781 | −5.33459 | −9.3624 |
| ENSRNOG00000023285 | Akr1b1-ps2 | −4.10603 | 4.475498 | 0.369467 |
| ENSRNOG00000053624 | AABR07058017.2 | −4.11829 | −2.76894 | −6.88723 |
| ENSRNOG00000068307 | Rn18s | −4.14182 | 0.206428 | −3.9354 |
| ENSRNOG00000052173 | AABR07008439.1 | −4.33103 | 4.344374 | 0.013339 |
| ENSRNOG00000065941 | NA | −4.33935 | 5.973977 | 1.63463 |
| ERCC-00098 | NA | −4.40124 | −2.21141 | −6.61265 |
| ENSRNOG00000049289 | Robo4 | −4.53504 | −3.80765 | −8.34269 |
| ENSRNOG00000068222 | LOC100910657 | −4.7402 | 5.709368 | 0.969169 |
| ENSRNOG00000042455 | Tlr12 | −4.93478 | −3.21836 | −8.15314 |
| ENSRNOG00000031486 | AABR07059215.1 | −5.71052 | 6.060307 | 0 |
| ENSRNOG00000064666 | NA | −6.12789 | 2.615695 | −3.51219 |

FIG. 23E illustrates a plot of relative gene expression of genes associated with neurons. The top genes reported in Table 3 include several genes that may be prevalently expressed within axons and dendrites, including Ncam2 which was the top differentially expressed gene (6.29-fold in coculture versus monoculture). Other highly expressed neuronal genes may include St6galnac5, Slc2a3, Grik2, and Tac1.

The influence of neurons on gene expression in co-cultures of epithelial cells and neurons may results from direct contact between the neurites and the epithelium. In some of the example embodiments, some of the top differential genes were related to neuronal function and found in the neuron extensions, like Ncam2. This may support a hypothesis that neurites extend through the porous membrane (1 μm pore size), for example, the porous membrane 326-1 described herein with reference to FIG. 3, into the epithelium culture. When mRNA was collected during the experiment, neural fragments may have been lifted from the top membrane as well.

FIG. 23F illustrates a plot of relative gene expression associated acetylcholine and VIP-related receptor genes.

With reference to FIG. 24, several of the top twenty enriched pathways were related to immune response. However, the example embodiments of an MPS described herein may lack the inclusion of specific immune cells. Adding complexity to the MPS by incorporating mast or dendritic cells may further advance the platform's ability to maintain homeostasis and achieve a closer match to what would be seen in the body. In addition, a few key genes related to cytokine production or inflammatory activation differed between the MPS and duodenal crypt samples, including Tnfa and Tlr4 gene expression. These genes were expressed at higher levels in both of the MPS samples compared to tissue. Such gene expression patterns may be due to the epithelium being heavily differentiated and undergoing anoikis when cultured in vitro. Anoikis, a programmed cell death that enterocytes undergo within the intestinal lining, may involve TNF-α reception with tumor necrosis factor receptor 1 (Tnfr1), often termed a death receptor due to the presence of a death domain. Example embodiments of in vitro monolayers may contain a greater ratio of cells undergoing anoikis compared to that found in vivo, which may result in heightened levels of Tnfa expression. A model that incorporates a self-renewing monolayer with a higher ratio of Lgr5 positive cells present may decrease the differential expression seen between MPS and tissue samples. Along with Tnfa, Tlr4 may be implicated in pro-inflammatory responses and anoikis via the NF-κB signaling pathway. This pathway promotes transcription of proinflammatory genes, including those for Il1b and Tnfa. NF-KB may also be involved in a cascade related to epithelial-mesenchymal transition (EMT) which may be activated via TLR4 and TNF-α signaling and causes epithelial cells to depolarize and detach from am extracellular matrix. The cells may then migrate and proliferate as needed to other sites. EMT is an important process related to stem cell maintenance, inflammation, and injury repair and could be activated in the example embodiments of monolayers in an MPS due to conditions of the microenvironment through factors such as cytokine production, altered WNT pathways, and growth factors. For example, decreased EGF levels may be identified in co-culture system via ELISA and increased Wnt2 expression, which may alter EMT when neurons are present, and therefore improve epithelial barrier integrity. Further validation of the behavior of co-cultures of different populations of cells, particularly including co-cultures interfacing in a desired 3-D arrangement or incorporating immune cells, that include more in-depth quantification of cytokines and growth factors may be achieved using embodiments of microfluidic devices and microphysiological systems described herein. Incorporating complex and branching architectures of the vascular and lymphatic systems may further influence cytokine expression and transport phenomena.

Modeling the duodenum using example embodiments of an MPS was chosen since it may be a heavily innervated part of the intestine and where a bulk of drug absorption occurs in humans or animals, and model duodenal systems may be underrepresented in current literature. However, duodenal organoids and monolayers may not as robust or well documented in literature as those from the colon. Colonic tissue may have a slower turnover rate and higher levels of mucus producing goblet cells. The slower turnover rate of colonic cells may translate into longer-lasting monolayer viability when compared to duodenal cells that will differentiate quickly and undergo apoptosis. The increased goblet cells, and as a result mucus barrier, of the colon may also provide a more substantial quantifiable barrier. Incorporating enteric neurons may be a valuable method for improving the quality of in vitro duodenal cultures, expanding the ability to study small intestine disorders.

Example embodiments of a microfluidic devices and microphysiological systems for cultures cells in 3-D arrangements have been described herein, including a model OOC of a small intestine using the example embodiments. Design improvements of example embodiments of the present invention may include increased throughput in, gravity driven flow, added tissue culture complexity (e.g., nerve innervation of co-cultures in a 3-D arrangement), and an example embodiment of an MPS focused on duodenal tissue. Experiments using the MPS focused on duodenal tissue may indicate that enteric neurons influence function and health of intestinal epithelium without an exogenous or inflammatory challenge. Cultures including the enteric nervous system and intestinal epithelium may include significantly stronger barrier function, changes in extracellular EGF, and increases in RNA encoding for cytokine and innate immunity genes. Such systems may provide improved tools and models for poorly understood disorders such as IBD and IBS, which consequently have insufficient treatment methods. The example embodiments described herein could further improve drug development and evaluation for such disorders. The experiments described herein my further demonstrate benefits of including more complex tissues, for example, more complex tissue populations and architectures, in GI models.

### Experimental Protocols

Protocols for example embodiments of the present invention, including microfluidic devices for the culturing of cells in a 3-D arrangement and microphysiological systems with cells cultured in a 3-D arrangement are described herein.

MPS Fabrication: Innervated MPSs were assembled layer-by-layer using laser cut thermoplastics with a previously established method. The system is scaled up to create a “high-throughput” design including twelve individual “chips” onto one device. Transparent, cast polymethyl methacrylate (PMMA) and clear 0.005-inch polyethylene terephthalate (PET) film may be used for the cell chamber wall layers. Tissue culture-treated PET, semi-permeable membranes with 1 um pores were utilized to separate the cell populations. Layered assembly may be achieved using pressure-sensitive transfer tape, and the chip was built on top of a glass slide base layer. Designs for each MPS layer may be created as vectors and cut using a laser cutter. Transfer tape was laminated onto the desired layers before laser cutting. MPSs were assembled by hand, heat pressed, and then placed into a vacuum oven for at least 120 h at 50° C. to allow curing and off-gassing of the adhesive material. Binder clips were used to apply additional pressure to the newly assembled MPS while baking.

Animal Care and Use: The following tissue isolation procedures were approved by Institutional Animal Care and Use Committee (IACUC). All methods were performed following the guidelines and regulations necessary.

Neuron Isolation and Cell Culture: Enteric neurons were isolated from neonatal, two-day-old Sprague-Dawley rats (p2) sacrificed via decapitation. Rat pups were mixed sex. The small intestines were removed, and the myenteric plexus was carefully peeled off the intestines using forceps under a dissecting microscope and stored in ice-cold Hanks Balanced Salt Solution (HBSS). The peeled myenteric plexus was moved into a 15 mL conical tube and digested for 1 h in collagenase type II and Deoxyribonuclease I dissolved in neurobasal medium at 37° C. The myenteric plexus in the digestion medium was vortexed for 5 s and examined visually. The digestion continued in 30-min increments until the myenteric plexus was visually broken up into small particles. Typically, this process is complete after three additional 30-min digestion steps. The solution was then centrifuged at 500 g for 5 min, and the supernatant was removed. The pellet was resuspended in a 0.05% trypsin-EDTA solution for 30 min at 37° C. to dissociate the neurons. After dissociation, the solution was centrifuged at 500 g for 5 min, and the trypsin supernatant was removed. The cell pellet was resuspended in enteric neuron medium (Table 1) and counted for culture. The fully digested neural tissue was seeded directly onto tissue culture treated glass, nonadherent well plates, or directly into the MPS (methods for gel encapsulation and seeding described later). Neurospheres were kept in suspension culture with enteric neuron media without the addition of any growth factors (NGF and GDNF), adherent or 3D cultures were fed the complete media as described in. Media was refreshed every 2-3 days either by half exchanging media every two to three days in adherent and 3D conditions or fresh media was added on top of the suspension cultures.

TABLE 4
Enteric neuron medium composition

| Medium Reagent | Supplier/Catalog # | Final Concentration |
|---|---|---|
| Neurobasal Medium | Gibco, 10888022 | 97% |
| Fetal Bovine Serum | Corning, 35015CV | 1% |
| Antibiotic/Antimycotic | Gibco, 15240-062 | 1% |
| Glutamax | Gibco, 35050061 | 1X |
| B27 Supplement | Gibco, 17504044 | 1X |
| GDNF | Gibco, PHC7045 | 10 ng/mL |
| NGF | R&D Systems, 256-GF/CF | 25 ng/mL |

Epithelial Organoid Isolation: Duodenal epithelial organoids were isolated from the first 3-4 cm of the small intestine of neonatal rat pups. After the myenteric plexus was removed from the segment, the remaining intestine was fileted open and cut into small pieces (˜5 mm). The diced intestines were washed ten times with cold Hanks balanced salt solution (HBSS). With the final wash, the HBSS was removed and replaced with 2 mM EDTA in HBSS. The tissue was left to digest on ice and rocking for 15 min. Then the tissue was allowed to settle, and the EDTA solution was removed and replaced with fresh 2 mM EDTA and digested for an additional 25 min on ice with rocking. When digestion was complete, crypts were detached from the rest of the tissue by vortexing the tube for 1 min, followed by pipetting vigorously using a 10 mL serological pipette while mixing about 25 times. The suspension was filtered through a 100 μm cell strainer and spun down at 300 g for 5 min. The supernatant was removed, and the pelleted crypts were resuspended in Advanced DMEM/F12 and spun again to wash, this was repeated three times. After the final wash and suspension removal, 20 μL of crypts were encapsulated in 1 mL growth factor reduced Matrigel and then plated into 50 μL domes in a 12-well plate, three domes per well. After the Matrigel had polymerized, the cells were fed an expansion medium (Table 5) with an addition of 10 μM rock inhibitor for the first feed after isolation or replating, Y-27632. Organoids were fed every 2-3 days with fresh expansion medium and passaged every 5-7 days.

TABLE 5
Expansion medium composition

| Medium | Supplier & | Working | | Final | | Volume | |
|---|---|---|---|---|---|---|---|
| Reagent | Catalog # | Concentration | | Concentration | | for 50 mL | |
| WRN Media | HDDC Organoid | N/A | | 50% | | 25 | mL |
| | Core/Breault Lab | | | | | | |
| Adv | Gibco, 12634028 | N/A | | 45% | | 22.5 | mL |
| DMEM/F12 | | | | | | | |
| Glutamax | Gibco, 35050061 | 100X | | 1X | | 500 | μL |
| HEPES | Fisher, | 1M | | 10 | mM | 500 | μL |
| | AAJ16924AE | | | | | | |
| Primocin | Invivogen, | 50 | mg/mL | 100 | μg/mL | 100 | μL |
| | NC9141851 | | | | | | |
| Normocin | Invivogen, | 50 | mg/mL | 100 | μg/mL | 100 | μL |
| | NC9273499 | | | | | | |
| B27 | Gibco, 17504-044 | 50X | | 0.5X | | 500 | μL |
| N2 | Gibco, A13707-01 | 100X | | 0.5X | | 250 | μL |
| N-Acetyl- | Sigma, A7250 | 500 | mM | 500 | μM | 50 | μL |
| Cysteine | | | | | | | |
| A-83-01 | Sigma, SML0788 | 500 | μM | 500 | nM | 50 | μL |
| SB202190 | Sigma, S7067 | 30 | mM | 10 | μM | 16.6 | μL |
| EGF | Peprotech, 315-09 | 50 | μg/mL | 5 | ng/mL | 5 | μL |
| Gastrin | Sigma, G9145 | 100 | μM | 10 | nM | 5 | μL |
| Y-27632 (only | Tocris Bioscience, | 10 | mM | 10 | μM | 50 | μL |
| added after | 12-541-0 | | | | | | |
| passaging) | | | | | | | |

Organoids were passaged by removing the media, replacing it with Cell Recovery Solution, and then disrupting the Matrigel domes by scraping them off the plate with a pipette tip. Organoids, Matrigel fragments, and cell recovery solution were collected into a tube and left on ice rocking for 45 min to degrade the Matrigel. Organoids were then spun down at 500 g for 5 min, and the supernatant was removed. At this point, 20 μL of the pellet was collected and resuspended in 1 mL of Matrigel, or the cells underwent further digestion for monolayer seeding.

MPS Seeding Protocol: Fabricated co-culture chips were sterilized by UV for 10 min, followed by oxygen plasma treatment for 90 s to prepare the membrane for protein coatings. The top membrane was coated with poly-d-lysine and incubated for 1 h at 37° C., and then rinsed with PBS at least twice. Next, a 1:10 dilution of Matrigel coating was added on top of the poly-d-lysine in preparation for epithelial monolayer adhesion. The MPS and Matrigel was incubated for at least 1 h at 37° C. before chips were seeded with the hydrogel and enteric neurons.

To seed organ chips with the neurospheres, the enteric neuron media with suspended neurospheres was first collected from the wells using a 1000 μL pipette and placed into a 15 mL tube. The tube was centrifuged at 300 g for 5 min to pellet the neurospheres. The supernatant was removed, and 1 mL of Accutase was added to the pellet. The Accutase solution was triturated to break up the pellet, and the tube was incubated for 30 min at 37° C. After incubation, the neurospheres were triturated 25-45 times using a 1 mL pipette tip and centrifuged at 300 g for 5 min. The Accutase was removed and replaced with fresh neuron medium, and cells were counted and seeded on-chip at 1×106 cells mL−1 in a 10% Matrigel, 2 mg mL−1 Cultrex Collagen solution in neuron media. The volume of cells needed to achieve 1×106 cells mL−1 was mixed with the Matrigel and Cultrex solution and 10 μL was injected into inlets on the MPS that lead into the enteric neuron chamber. The gels were thermally crosslinked at 37° C. for 20 min before medium was added to the reservoirs of the chip. Neurons received a complete media exchange after 24 h with 20 μM of the mitotic inhibitor, cytosine β-D-arabinofuranoside. During the remaining timeframe, the neurons received half-volume media exchanges.

After a week of neuron culture maturation, we seeded the epithelial monolayer. The Matrigel domes were disrupted with manual scraping using a pipette tip. Organoids and Matrigel fragments were collected in cell recovery solution and rocked on ice for 45 min to remove the organoids from the Matrigel. Organoids were centrifuged at 500 g for 5 min, and the cell recovery solution was removed. Organoids were resuspended in trypLE and digested for 5 min at 37° C. The cells were then agitated by pipetting up and down through a bent 1 mL pipette tip 20 times to achieve a single-cell suspension. Cells were spun down to remove the trypLE, resuspended in the expansion medium with 10 μM Y-27632 rock inhibitor, and counted. Chips were seeded with 600,000 cell per chip, 2.2×106 cells cm⁻². After one day, the media was changed out for differentiation medium (Table 6).

TABLE 6
Epithelial differentiation medium composition

| Medium | Supplier & | Working | | Final | | Volume | |
|---|---|---|---|---|---|---|---|
| Reagent | Catalog # | Concentration | | Concentration | | for 10 mL | |
| Advanced | Gibco, 12634028 | N/A | | 85% | | 8.5 | mL |
| DMEM/F12 | | | | | | | |
| R-Spondin | HDDC Organoid | N/A | | 10% | | 1 | mL |
| | Core/Breault Lab | | | | | | |
| Glutamax | Gibco, 35050061 | 100X | | 1X | | 100 | μL |
| HEPES | Fisher, | 1M | | 10 | mM | 100 | μL |
| | AAJ16924AE | | | | | | |
| Primocin | Invivogen, | 50 | mg/mL | 100 | μg/mL | 20 | μL |
| | NC9141851 | | | | | | |
| Normocin | Invivogen, | 50 | mg/mL | 100 | μg/mL | 20 | μL |
| | NC9273499 | | | | | | |
| B27 | Gibco, 17504-044 | 50X | | 0.5X | | 100 | μL |
| Supplement | | | | | | | |
| N2 Supplement | Gibco, A13707-01 | 100X | | 0.5X | | 50 | μL |
| N-Acetyl- | Sigma, A7250 | 500 | mM | 1.25 | mM | 10 | μL |
| Cysteine | | | | | | | |
| EGF | Peprotech, 315-09 | 50 | μg/mL | 5 | ng/mL | 1 | μL |
| Noggin | Peprotech, 250-38 | 100 | μg/mL | 100 | ng/mL | 10 | μL |
| Y-27632 | Sigma, Y0503 | 10 | mM | 10 | uM | 10 | μL |
| (Rock | | | | | | | |
| Inhibitor) | | | | | | | |
| A-83-01 | Sigma, SML0788 | 500 | μM | 500 | nM | 10 | μL |
| SB202190 | Sigma, S7067 | 30 | mM | 10 | μM | 3.3 | μL |
| Gastrin | Sigma, G9145 | 100 | μM | 10 | nM | 1 | μL |
| Valproic Acid | Sigma, P4543 | 1M | | 1 | mM | 10 | μL |
| CHIR99021 | Sigma, SML1046 | 10 | mM | 3 | μM | 3 | μL |

Media Compositions: According to supplier suggestions, lyophilized reagents were resuspended to their working concentration in deionized water, DMSO, or PBS. Rat enteric neuron medium was used for feeding neuron cultures (Table 4), with a half volume exchange performed every two to three days. Rat organoid proliferation medium (Table 5) was used during organoid expansion in Matrigel domes and for the first 24 h of seeding a monolayer. For chip monolayer experiments, after 24 h, the epithelial medium reservoir was exchanged for rat organoid differentiation medium (Table 6) to encourage differentiation into representative heterogeneous cell populations.

Application of Shear: MPSs were placed on a platform rocker in an incubator with 5% CO2 at 37° C. for three days. A rocker tilt angle of 2° and rotation speed of 10 rpm were determined to generate a physiological rate of shear force (0.002-0.08 dyne cm⁻²), calculated at the center of the monolayer culture surface area when at the maximum tilt angle. Calculations were determined using the assumptions and formulas stated previously reported. The critical flip angle, θ₀, when the fluid-free surface first contacts the edge of the bottom of the MPS, was calculated using the MPS's epithelial channel dimensions (Equation 1). Here, h₀ represents the medium depth, and L represents the length of the epithelial channel. Once the critical flip angle was determined, the shear stress could be calculated using Equation 2, where θ is the rocking angle, θ₀ is the critical flip angle, T is the period, and x is the location of interest within the channel.

Lucifer Yellow Permeability Assay: Lucifer yellow lithium salt was resuspended to a 100 μM stock solution in PBS and wrapped in foil to avoid light exposure. A 10 mM working solution was prepared in phenol red free DMEM. All media was carefully removed from apical and basal chip compartments, and 150 μL of 10 mM lucifer yellow was added into the apical compartments of all the chip wells. Phenol red free DMEM was added to the basal compartments (150 μL) as well. The chips were incubated at 37° C. for 3 h at static conditions. After incubation, 100 μL of the solution was removed from the apical and basal channels and put into separate wells of a black 96-well plate. A standard curve for the lucifer yellow working solution was prepared using 1:2 serial dilutions of the 10 mM solution and phenol red free DMEM. The fluorescence was then measured for each well on a plate reader spectrophotometer using 428/530 excitation/emission settings with the gain set between 50 and 75. Apparent permeability was then calculated using the concentration values determined from a linear fit of the standard curve.

Immunostaining: Cells were fixed with paraformaldehyde for 10 min. After fixation, the solution was removed from the culture chambers, and a rinsed with HBSS twice. Cells were permeabilized in a 0.1% Tween-20 solution for 10 min. After rinsing out the Tween, a blocking solution of filtered 4% goat serum was added to the culture and left to incubate overnight at 4° C. The following day, the blocking solution was removed. Antibodies (Table 7) diluted in 4% goat serum were added into the wells and incubated for 2 h at room temperature or overnight at 4° C. Following antibody addition, MPS compartments were carefully rinsed with HBSS three times. Secondary antibodies diluted in 4% goat serum were then added and incubated for 2 h at room temperature or overnight at 4° C. Following secondary incubation, DAPI was added at 1:1000 dilution in PBS for 10 min. Rinsing was then performed carefully in the MPS at least three times. MPSs were imaged immediately or stored at 4° C. and wrapped with parafilm. Terminal Epithelial Monolayer Characterization: Monolayers were stained for filamentous actin with Phalloidin to identify the cell cytoskeleton and total area. Tiled images of the entire culture area of the immunostained MPSs were acquired on a Zeiss Axio Observer Z1. Exported images were analyzed using Fiji in ImageJ. Briefly, the images were cropped to a uniform size matching the dimensions of the chip culture area. Then a variance filter was applied to improve contrast at the edges of the cells. The MorphoLibJ plugin was used to segment the image, and the total cell area covered was recorded.

TABLE 7
Antibodies and dilutions for immunohistochemistry

| | Catalog | | ICC | |
|---|---|---|---|---|
| Antibody | ID/Supplier | Host | Dilution | Notes |
| Beta III Tubulin | Sigma, T8660 | Mouse | 1:1000 | Structural, |
| | | | | mature |
| | | | | neurons |
| Zonula | Invitrogen, | Rabbit | 1:100- | Tight |
| Occludens-1 | 40- | | 1:200 | junctions, |
| (ZO-1) | 2200 | | | mature |
| | | | | epithelium |
| Vasoactive | Abcam, | Rabbit | 1:200 | VIP producing |
| Intestinal Peptide | AB8556 | | | neurons |
| (VIP) | | | | |
| Choline | Millipore, | Chicken | 1:1000 | Acetylcholine |
| Acetyltransferase | AB15468 | | | producing |
| (ChAT) | | | | neurons |
| Phalloidin Alexa | Invitrogen, | NA | 1:1000 | F-actin |
| Fluor 647 | A22287 | | | |
| Anti-Chicken, | Invitrogen, | Goat | 1:1000 | Secondary |
| Alexa Fluor 488 | A11039 | | | |
| Anti-Rabbit, | Invitrogen, | Goat | 1:1000 | Secondary |
| Alexa Fluor 546 | A11035 | | | |
| Anti-Mouse, | Invitrogen, | Goat | 1:1000 | Secondary |
| Alexa Fluor 647 | A32728 | | | |

Acetylcholine and Vasoactive Intestinal Peptide Neuron Subtype Quantification: Neonatal rat enteric neurons were isolated as described above and plated on cover glasses. Cover glasses were sterilized by UV for 5 min on each side, oxygen plasma treated for 90 s, poly-d-lysine coated for 1 h followed by washes, and finally 50 μg mL−1 mouse laminin coating for 30 min. Neurons were seeded at a density of 50,000 cells per well in a 24-well plate with a half-exchange medium feeding every two to three days. After 1 week, the neurons were fixed with 4% paraformaldehyde for 10 min and then washed with HBSS 3 times. The cells were permeabilized with 0.1% Triton-X for 10 min, followed by additional washes with HBBS. Blocking was done with 2.5% goat serum for at least 1 h before adding the primary antibodies for Beta III Tubulin, VIP, and ChAT (Table 7). Primary antibodies were incubated at room temperature for at least 1 h, followed by 3 washes with HBSS. The secondary antibody solution was similarly made in 2.5% goat serum and incubated at room temperature for at least 1 h, followed by 3 washes. Cover glasses were then mounted on microscope slides with ProLong Gold Antifade Mountant and DNA Stain DAPI.

Images of the stained neurons were segmented and quantified using Cell Profiler. Regions of interest were segmented using the nuclei stain of DAPI as a seed and Beta III Tubulin for the boundaries of the cell. Individual cells were quantified for their integrated intensity, the sum of pixel intensities per region of interest. They were considered positively stained for VIP or ChAT if they were greater than 1 standard deviation from the mean of all the neurons imaged per experimental replicate. Immunostained samples were imaged on a Zeiss Axio Observer Z1 for the MPS z-stacks, MPS orthogonal projections, neuronal subtype and phalloidin cell coverage experiments.

Transepithelial Electrical Resistance (TEER) Measurements: TEER was performed using a World Precision Instruments EVOM device with chopstick electrodes. Electrodes were first cleaned in 70% ethanol and calibrated in PBS. Recordings were made by completely submerging the electrodes into the media of the apical and basal chambers and allowing the resistance reading to equilibrate. A recording was taken for each basal inlet of each sample and averaged. TEER calculations were then normalized to the area of the chip (0.49 cm²) to get Q cm-2. ELISA Assays: EGF and Muc2 Secretion: At experiment end points, cell culture supernatants were collected separately from each sample's apical and basal compartments and centrifuged at 3000 rpm for 10 min at 4° C. The centrifuged supernatants were moved into a clean microcentrifuge tube and stored immediately at −80° C. until needed. An EGF ELISA was performed on the collected basal supernatants following supplier instructions with samples ran in duplicate and averaged. A Muc2 ELISA was performed similarly using the apical supernatants. Optical densities were obtained using a plate reader at 620 nm and compared to a standard curve fit.

RNA Sequencing Methods: Epithelial cells monocultured and cocultured in the MPS devices were lifted from the membrane using 0.25% trypsin-EDTA. Cells were incubated in trypsin-EDTA for 10-min increments and agitated via pipetting to release them from the membrane. The culture area was checked under the microscope after each 10-min incubation to ensure the majority of the cells had been lifted. Cells were then pipetted into a microcentrifuge tube and centrifuged at 500 g for 5 min to form a pellet. The cell pellets were rinsed with HBSS and spun again. HBSS was removed and cell pellets were stored dry at −80° C. until shipment. Three samples of epithelial cells for each condition (monoculture and co-culture with neurons) were pooled into two independent samples. Pooling allowed adequate collection of RNA since MPS chamber volumes are small. Next generation sequencing (NGS) is performed on the pooled samples. RNA was extracted by a third-party vendor and appropriate quality checks were performed. Low input RNA sequencing methodology was utilized using an Illumina System and the raw counts with gene IDs were provided by the supplier. Differential and relative gene expression, as well as top enriched pathways were quantified using the raw count files imported into iDEP.96. A log 2-fold change method was used for differential expression and normalized enrichment scores (NES) and Reactome pathways were used for pathway enrichment analysis.

Statistical Analyses: GraphPad Prism was used to run statistical testing. Normality tests were performed on each data set. Data sets with more than two experimental trials were analyzed for statistical significance (p<0.05) using an unpaired t-test or one-way ANOVA with multiple comparisons for normally distributed data sets and a Kruskal-Wallis nonparametric test for non-normal data sets. All experiments, excluding the phalloidin area coverage and RNA sequencing, were performed independently in triplicate. For experiments using primary cells, litters of 10 mixed-sex rat pups were used and pooled per experimental trial.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A fluidic device for culturing cells in a three-dimensional (3-D) arrangement, the fluidic device comprising:
a plurality of layers including:
a first layer configured to define a first chamber for holding a first cell culture;
a second layer configured to define a second chamber for holding a second cell culture, the first layer and the second layer in coupled arrangement to fluidically couple the first chamber and the second chamber and to enable the first cell culture and the second cell culture to grow in the 3-D arrangement;
a porous membrane positioned between the first layer and the second layer and configured to enable interfacing between at least a portion of the first cell culture and at least a portion of the second cell culture through the porous membrane; and
one or more channel layers configured to define one or more channels, each channel layer in coupled arrangement with the first layer or the second layer to fluidically couple each channel with at least one of the first chamber or the second chamber, wherein the one or more channels are configured to enable passage of one or more fluids with respect to the cells cultured in the 3-D arrangement.

2. The fluidic device of claim 1, wherein the plurality of layers is configured to define one or more ports fluidically coupled to the one or more channels, the first chamber, or the second chamber.

3. The fluidic device of claim 2, further comprising a reservoir layer configured to define at least one media reservoir, a given media reservoir fluidically coupled with a channel of the one or more channels through a port of the one or more ports.

4. The fluidic device of claim 3, wherein the given media reservoir is configured to dispense the one or more fluids into the channel based on an orientation of the fluidic device, the dispensing of the one or more fluids imparting shear stress on the first cell culture or the second cell culture.

5. The fluidic device of claim 2, wherein a port of the one or more ports is fluidically coupled with a pump, the pump configured to supplement media continuously or to cause the passage of the one or more fluids through a channel of the one or more channels.

6. The fluidic device of claim 2, wherein the one or more channel layers includes a first channel layer configured to define a first channel, the first channel fluidically coupled to the first chamber, and a second channel layer configured to define a second channel, the second channel fluidically coupled to the second chamber, further wherein the one or more ports include at least a first port fluidically coupling the first channel to a given surface of the fluidic device and at least a second port fluidically coupling the second channel to the given surface of the fluidic device.

7. The fluidic device of claim 1, further comprising an additional porous membrane configured to be selectively permeable, the additional membrane positioned between a channel of the one or more channels and the first layer or the second layer.

8. The fluidic device of claim 1, further comprising the first cell culture and the second cell culture.

9. The fluidic device of claim 1, wherein the plurality of layers are configured to enable imaging of the cells in situ.

10. The fluidic device of claim 1, wherein a given layer of the plurality of layers is bonded to another given layer of the plurality of layers.

11. A high throughput fluidic system for culturing cells in a 3-D arrangement, the high throughput fluidic system comprising:
the plurality of layers of claim 1, wherein:
the first chamber defined by the first layer, the second chamber defined by the second layer, and the one or more channels defined by the one or more channel layers are a first fluidic unit, the plurality of layers further configured to define at least one additional fluidic unit.

12. The fluidic system of claim 11, wherein the plurality of layers is configured to define one or more ports fluidically coupled to the one or more channels, the first chamber, or the second chamber of a given fluidic unit of the first fluidic unit or the at least one additional fluidic unit.

13. The fluidic system of claim 12, further comprising a reservoir layer configured to define a plurality of media reservoirs, a given media reservoir fluidically coupled to a given port of the one or more ports and configured to dispense a fluid into a given channel of the one or more channels based an orientation of the high throughput fluidic system.

14. The fluidic system of claim 11, wherein a given fluidic unit of the first fluidic unit or the at least one additional fluidic unit is fluidically independent of another fluidic unit of the first fluidic unit or the at least one additional fluidic unit.

15. A physiological fluidic system with cells cultured in a three-dimensional (3-D) arrangement, the physiological fluidic system comprising:
a first layer configured to define a first chamber, the first chamber including a first cell culture;
a second layer configured to define a second chamber, the second chamber including a second cell culture, wherein the first layer and the second layer are in coupled arrangement to fluidically couple the first chamber and the second chamber and to enable the first cell culture and the second cell culture to grow in the 3-D arrangement;
a porous membrane positioned between the first layer and the second layer and configured to enable interfacing between at least a portion of the first cell culture and at least a portion of the second cell culture through the porous membrane; and
one or more channel layers configured to define one or more channels, the one or more channel layers in coupled arrangement with the first layer or the second layer to fluidically couple the one or more channels with the first chamber or the second chamber, wherein the one or more channels are configured to enable passage of one or more fluids with respect to the cells cultured in the 3-D arrangement.

16. The physiological fluidic system of claim 15, wherein the first cell culture or the second cell culture includes epithelial cells or endothelial cells and the other of the first cell culture or the second cell culture includes neurons.

17. The physiological fluidic system of claim 15, further comprising a structural hydrogel for the first cell culture in the first chamber or for the second cell culture in the second chamber.

18. A method for culturing cells in a three-dimensional (3-D) arrangement in a fluidic system, the method comprising:
seeding a first cell culture in a first cell chamber;
seeding a second cell culture in a second cell chamber, the first cell chamber and the second cell chamber being fluidically coupled and being separated by a porous membrane, the first cell chamber and the second cell chamber further positioned to enable the first cell culture and the second cell culture to grow in the 3-D arrangement;
culturing the first cell culture and the second cell culture to enable interfacing of at least a portion of the first cell culture and at least a portion of the second cell culture through the porous membrane; and
causing passage of a fluid with respect to the cells cultured in the 3-D arrangement.

19. The method of claim 18, wherein the passage of the fluid includes causing a flow of the fluid at a surface of at least a portion of the first cell culture or at least portion of the second cell culture, the flow causing sheer stress on surface.

20. The method of claim 18, wherein the first cell culture is seeded and cultured for a period of time prior to seeding of the second cell culture.
